(19) United States
(12) Patent Application Publication
(10) Pub. No.: US 2018/0154030 A1
(43) Pub. Date: Jun. 7, 2018

(54) System and Method For Sterilizing and/or Deimmunizing an Object

(71) Applicant: ONEighty°C Technologies Corporation, Burlington, MA (US)

(72) Inventors: John Erickson, Middleton, MA (US); Theresa L. O'Keefe, Waltham, MA (US); Eric Walters, Concord, MA (US); Kevin Staid, Redwood City, CA (US)

(21) Appl. No.: 15/792,148

(22) Filed: Oct. 24, 2017

Related U.S. Application Data
(63) Continuation-in-part of application No. 15/330,469, filed on Sep. 23, 2016.
(60) Provisional application No. 62/232,055, filed on Sep. 24, 2015.

(51) Int. Cl. A61L 2/12; A61L 2/24; A61L 2/18

(52) U.S. Cl. CPC A61L 2/12; A61L 2/24; A61L 2202/17; A61L 2202/14; A61L 2202/24; A61L 2/18

(57) ABSTRACT

A system for sterilizing and/or deimmunizing an object includes a stationary chamber at ambient pressure is configured to store an object to be sterilized and/or deimmunized therein. A solvent delivery subsystem is coupled to the chamber and configured to apply a directed volume of a non-toxic aqueous solvent to coat and wet the object to optimally hydrate proteins of infectious agents and/or immunological agents in or on the object for proteolysis. An electromagnetic device is coupled to the chamber and is configured to direct microwaves at the object to induce proteolysis of the proteins and generate heat to dry the non-toxic aqueous solvent in or on the object in or on the object. A temperature control subsystem is coupled to the chamber and is configured to control the temperature of the chamber to induce a temperature of the proteins which accelerates proteolysis of the proteins and dries the non-toxic aqueous solvent in or on the object. A controller subsystem coupled to the solvent delivery subsystem, the electromagnetic device and the temperature control subsystem is configured to provide a wet/dry cycle including activating the solvent delivery subsystem for a predetermined amount of time to wet and hydrate the proteins for proteolysis, activating the electromagnetic device for a predetermined amount of time to induce proteolysis and dry the non-toxic aqueous solvent in or on the object, and activating the temperature control system a predetermined amount of time to accelerate the proteolysis of the proteins and dry the non-toxic aqueous solvent in or on the object, and repeating the wet/dry cycle a predetermined number of times to irreversibly destroy proteins in or on the object to sterilize and/or deimmunize the object.

### RELATED APPLICATIONS

This application is a continuation-in-part of U.S. patent application Ser. No. 15/330,469 filed Sep. 23, 2016 and claims the benefit of and priority thereto under 35 U.S.C. §§ 119, 120, 363, 365, and 37 C.F.R. § 1.55 and § 1.78, and U.S. patent application Ser. No. 15/330,469 claims the benefit of and priority to U.S. Provisional Application Ser. No. 621232,055 filed Sep. 24, 2015 under 35 U.S.C. §§ 119, 120, 363, 365, and 37 C.F.R. § 1.55 and § 1.78, and each of these applications is incorporated herein by this reference.

### FIELD OF THE INVENTION

This invention relates to a system and method for sterilizing and/or deimmunizing an object.

### BACKGROUND OF THE INVENTION

As medical treatments and diagnostics move away from traditional large incision processes, medical devices have become more flexible and complicated. To enable flexibility and/or smaller incisions, medical devices often use non-metal materials including a wide range of plastics, and the like. Additionally, the equipment frequently includes articulating joints and narrow lumens.

Medical devices composed of plastics with articulating joints and narrow lumens are frequently not robust enough to survive the rigors of conventional autoclave sterilization methods. As a result, they can only be prepared for reuse by vigorous cleaning and disinfection processes that frequently leave behind, including, inter alia, infectious and/or immunogenic agents defined herein as infectious proteins, spore forming bacteria, vegetative bacteria, funguses, mix of bacteria protected by a biofilm, infectious or immunogenic proteins, and toxic proteins that can infect and injure patients who are later treated using the insufficiently sterilized medical equipment.

Some of these infectious agents can be effectively eliminated through thorough cleaning and disinfection of medical equipment. Other infectious agents are extremely difficult to eliminate from medical equipment. The Center for Disease Control (CDC) lists examples of infectious agents and microorganisms by resistance to disinfection and sterilization processes. See Table 1 below:

TABLE 1
Decreasing order of resistance of infectious agents and
microorganisms to disinfection and sterilization

| Agent Category | Example Organisms or Diseases |
|---|---|
| Prions | Creutfeldt-Jakob Disease |
| Bacterial spores | Bacillius atrophaeus |
| Coccidica | Cryptosporidium |
| Mycobacteria | M. tuberculosis, M. terrae |
| Nonlipid or small viruses | polio, coxsackie |
| Fungi | Aspergillus, Candida |
| Vegetative bacteria | S. aureus, P. aeruginosa |
| Lipid of medium-sized viruses | HIV, herpes, hepatitis B |

### (CDC's Guideline for Disinfection and Sterilization in Healthcare Facilities, 2008)

Some infectious agents, such as the HIV virus, are easy to remove from medical equipment. Many, including vegetative bacteria are moderately difficult to eliminate. Other infectious agents, such as prions, can only be destroyed by extremely harsh conditions that damage and/or destroy modem medical equipment. Failure to eliminate infectious agents from medical equipment before use can put patients at extreme risk of injury and death.

Sterilization is a physical or chemical process that completely destroys or removes all forms of infectious agents from an object, including spore forming bacteria. Such spores allow the bacteria to resist high temperatures and other harsh conditions. Although measured in Sterility Assurance Levels (SAL), sterility is an absolute condition, i.e. either an item is sterile or it is not. Disinfection is a process that eliminates many or all infectious agents on instruments with the exception of bacterial spores. Disinfection is not absolute and is classified into three different levels: 1) High-Level Disinfection: kills all microorganisms with the exception of many bacterial spores through the use of chemical sterilants used for a shorter exposure period than would be required for sterilization, 2) Intermediate-Level Disinfection: may kill mycobacteria, vegetative bacteria, most viruses, and most fungi but do not necessarily kill bacterial spores, and 3) Low-level disinfection: may kill most vegetative bacteria, some fungi, and some viruses.

Although vegetative bacteria may be only moderately difficult to eliminate, many vegetative bacteria are still found to contaminate medical equipment after cleaning and disinfection. In addition to causing disease in patients, a number of species have been found to carry genes that allow the bacteria to grow and remain infectious even during the patients' treatment with antibiotics. Examples include, inter alia, Clostridium difficile (C. diff), a, CRE (Carbapenem-resistant Enterobacteriace) and MRSA (Methicillin-resistant Staphylococcus aureus) that are resistant to many antibiotics and in a medical setting can cause severe intestinal infection and life-threatening bloodstream infections, pneumonia and surgical site infections.

Prions (PrP—protease resistant proteins) are a unique category of transmissible infectious agent that causes a wide range of diseases including new variant Creutzfeldt-Jakob Disease. As Prions are only protein and do not include DNA or RNA, their destruction may be termed deactivation instead of sterilization. Prions are an abnormally folded protein (PrP_{sc}) that cause disease symptoms by promoting the unfolding of the normal protein (PrP_{c}) and refolding into the disease causing form (PrP_{sc}). With most infectious agents, conventional heat or steam systems and methods are sufficient to render the agents permanently non infections. However, such conventional heat and steam methods are unable to eliminate infectious prions from medical equipment.

When determining what level of sterilization or disinfection is appropriate for a particular reusable medical instrument or equipment, the Centers for Disease Control and Preventions (“CDCP”) uses a classification scheme which categorizes items, such as medical instruments and equipment, as either critical, semi-critical, or non-critical according to the degree of risk of infection being introduced by their use if not properly sterilized. Critical items represent the highest level of risk of infection if contaminated with any microorganism. Examples include medical instruments and equipment that enter tissue or the vascular system and include surgical instruments and equipment, cardiac and urinary catheters, implants and ultrasound probes used in body cavities. Medical instruments and equipment must by sterilized between uses. Semi-critical items, such as medical instruments and equipment, represent the next highest level of risk of infection are items that contact mucous membranes, such as the mucous membrane of the lungs or gastrointestinal tract. Semi-critical items are generally less likely to transfer common bacterial spores between patients but are highly susceptible to be able to transfer other organisms, such as bacteria, mycobacteria, and viruses. Semi-critical items require minimal high-level disinfection. While laparoscopes and arthroscopes should ideally be sterilized, they sometimes undergo a semi-critical level disinfection between patients.

Non-critical items, such as medical instruments and equipment that contact the skin but not mucous membranes, represent the least of risk for the transfer of infection between patients. Examples used in patient care include blood pressure cuffs, bedpans, crutches, and the like, and other related items.

Using a combination of one or more of heat, steam, water and microwaves to sterilize and/or disinfect medical equipment is known in the art, e.g., as disclosed in U.S. Pat. No. 6,900,421, incorporated herein by reference. The '412 Patent teaches a complicated and cumbersome system which must be pressurized by requiring a sealed first chamber capable of withstanding internal pressure and vacuum, generating steam greater than 1 atmosphere, introducing steam into the chamber, and removing the steam or by displacing it with nitrogen.

Another conventional apparatus for heating, disinfecting and sterilizing materials using microwave radiation, heat and water is disclosed in U.S. Pat. No. 5,879,643, incorporated by reference herein. As disclosed therein, a microwave device radiates microwave energy at refuse inside a container located in a chamber. The '643 Patent also teaches using a spray system with heated water which moistens the material being treated. The goal of the '643 Patent is to use water to eliminate the risk of fire which may result from using microwaves which may excessively heat the water.

U.S. Pat. Nos. 7,507,369, 7,687,045, and 7,939,016 now owned by the assignee hereof, teach another complicated and cumbersome system for disinfecting and/or sterilizing mail. As disclosed therein, mail to be disinfected is placed in a rotating drum and subjected to a source of radiation, microwaves, ultraviolet radiation, and a chemical decontamination unit.

All of the conventional systems discussed above which utilize one or more of microwaves, water, steam, and/or heat fail to teach or disclose irreversibly destroying proteins which are components of infectious and/or immunogenic agents, including, inter alia, bacterial spores, vegetative bacteria, viruses, funguses, infectious or immunogenic proteins, and toxic proteins to sterilize and/or deimmunize an object.

### SUMMARY OF THE INVENTION

In one aspect, a system for sterilizing and/or deimmunizing an object is featured. The system includes a stationary chamber at ambient pressure configured to store an object to be sterilized and/or deimmunized therein. A solvent delivery subsystem coupled to the chamber is configured to apply a directed volume of a non-toxic aqueous solvent to coat and wet the object to optimally hydrate proteins of infectious agents and/or immunological agents in or on the object for proteolysis. An electromagnetic device coupled to the chamber configured to direct microwaves at the object to induce proteolysis of the proteins and generates heat to dry the non-toxic aqueous solvent in or on the object in or on the object. A temperature control subsystem coupled to the chamber is configured to control the temperature of the chamber to induce a temperature of the proteins which accelerates proteolysis of the proteins and dries the non-toxic aqueous solvent in or on the object. A controller subsystem coupled to the solvent delivery subsystem, the electromagnetic device and the temperature control subsystem are configured to provide a wet/dry cycle including activating the solvent delivery subsystem for a predetermined amount of time to wet and hydrate the proteins for proteolysis, activating the electromagnetic device for a predetermined amount of time to induce proteolysis of the proteins and dry the non-toxic aqueous solvent in or on the object, and activating the temperature control subsystem a predetermined amount of time to accelerate the proteolysis of the proteins and dry the non-toxic aqueous solvent in or on the object, and repeating the wet/dry cycle a predetermined number of times to irreversibly destroy proteins in or on the object to sterilize and/or deimmunize the object.

In one embodiment, the temperature control subsystem may be configured to maintain a temperature in the chamber which does not damage the object. The object may include a gastrointestinal scope. The gastrointestinal scope may include one of: a duodenoscope, an endoscope, or a colonoscope. The object may include a lumen in the duodenoscope, the endoscope, or the colonoscope. The object may include a biofilm on the lumen. The object may include a movable tip of the duodenoscope. The electromagnetic device may be activated a predetermined amount of time to heat the chamber, the object, and the proteins to a predetermined range of temperatures. The temperature control subsystem may include one or more heating devices coupled to the chamber configured to heat the chamber, the object, and the proteins to a predetermined range of temperatures. The system may include one or more cooling devices coupled to the chamber configured to cool the chamber, the object, and the proteins to a predetermined range of temperatures. The system may include a positioning and/or storage device inside the chamber configured to secure and position the object in the positioning and/or storage device to increase the proteolysis and the irreversible destruction of proteins in or on the object to sterilize and/or deimmunize the object. The positioning and/or storage device may include at least one area of a dielectric material positioned proximate a predetermined area of the object, the dielectric material configured to focus the microwaves at the predetermined area of the object to further increase the proteolysis and the irreversible destruction of proteins to sterilize and/or deimmunize the object and to enhance drying the non-toxic aqueous solvent at the predetermine area of the object. The system may include a susceptor coupled to the dielectric material configured to further focus the microwaves at the predetermined area of the object to further increase the proteolysis and the irreversible destruction of the proteins to sterilize and/or deimmunize the object and enhance drying of the non-toxic aqueous solvent in or on the object. The positioning and/or storage device may include an electromagnetic shield housing configured to house a predetermined portion of the object and minimize microwaves from contacting and damaging the portion of the object. The positioning and/or storage device may include a tray for securing and positioning the object and a cover. The cover may include one or more ports coupled to the solvent delivery subsystem configured to apply the non-toxic aqueous solvent in or on the object. The positioning and/or storage device may include one or more openings configured to receive a line coupled to the solvent delivery system, the line configured to apply the non-toxic aqueous solvent to a lumen of the object. The lumen of the object may include the lumen of a duodenoscope, an endoscope, or a colonoscope. The object may include a biofilm on the lumen. The positioning and/or storage device may be configured to store a sterilized and/or deimmunized object in a sterile condition for a predetermined amount of time. The non-toxic aqueous solvent may include one or more of: water, an alcohol solution, hydrogen peroxide, an ionic detergent, and/or a non-ionic detergent. The proteins may be components of an infectious and/or immunological agents including spore forming bacteria, vegetative bacteria, viruses, funguses, a mix of bacteria protected by a biofilm, infectious or immunological proteins, and toxic proteins. The electromagnetic device and the chamber may be configured as a modified microwave device.

In another aspect, a method for sterilizing and/or deimmunizing an object is featured. The method includes providing a stationary chamber at ambient pressure configured to store an object to be sterilized and/or deimmunized therein, applying a directed volume of a non-toxic aqueous solvent to coat and wet the object to optimally hydrate proteins of infectious agents and/or immunological agents in or on the object for proteolysis, directing microwaves at the object to induce proteolysis of the proteins and generate heat to dry the non-toxic aqueous solvent in or on the object, controlling the temperature of the chamber such that the temperature of the proteins in or on the object accelerates proteolysis of the proteins in or on the object and dries the non-toxic aqueous solvent in or on the object, and providing a wet/dry cycle including coating and wetting the proteins to optimally hydrate the proteins applying microwaves a predetermined amount of time to induce proteolysis of the proteins and dry the non-toxic aqueous solvent in or on the object, controlling the temperature a predetermined amount of time to accelerate proteolysis of the proteins and dry the non-toxic aqueous solvent in or on the object, and repeating the wet/dry cycle a predetermined number of times to irreversibly destroy proteins in or on the object to sterilize and/or deimmunize the object.

In one embodiment, the controlling the temperature may include maintaining a temperature in the chamber which does not damage the object. The object may include a gastrointestinal scope. The gastrointestinal scope may include one of: a duodenoscope, an endoscope, or a colonoscope. The object may include a lumen in the duodenoscope, the endoscope, or the colonoscope. The object may include a movable tip of the duodenoscope. The object may include a biofilm on the lumen. Controlling the temperature of the chamber may include providing a predetermined range of temperatures in the chamber. Directing the microwaves may include focusing the microwaves on a predetermined area of the object. The method may include minimizing the microwaves from contacting a portion of the object. The non-toxic aqueous solvent may include one or more of: water, an alcohol solution, hydrogen peroxide, an ionic detergent, and/or a non-ionic detergent. The proteins may be components of an infectious and/or immunological agents including spore forming bacteria, vegetative bacteria, viruses, funguses, a mix of bacteria protected by a biofilm, infectious or immunological proteins, and toxic proteins.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic block diagram showing the primary components of one embodiment of the system for sterilizing and/or deimmunizing an object;

FIG. 2 is a three-dimensional front view of the system shown in FIG. 1 configured as a modified microwave oven;

FIG. 3 is a three-dimensional view showing the inside of the modified microwave oven shown in FIG. 2;

FIG. 4 is a schematic circuit diagram showing in further detail the primary components of the controller subsystem shown in FIGS. 1 and 2;

FIG. 5 is a screen shot showing one example of the various parameters controlled by the controller subsystem shown in FIGS. 1 and 2;

FIG. 6 is a flow chart showing the primary steps associated with one embodiment of the method for sterilizing and/or deimmunizing an object;

FIGS. 7A and 7B show an example of a filter strip having an infectious and/or immunogenic agent thereon to be sterilized and/or deimmunized using the system and method shown in FIGS. 1-6;

FIG. 8 shows an example of a Western Analysis for sample shown in FIG. 7B sterilized and/or deimmunized using the system and method shown in FIGS. 1-6:

FIG. 9 shows an example of another filter strip having a different infectious agent thereon to be sterilized and/or deimmunized using the system and method shown in FIG. 1-6; and

FIG. 10 is a three-dimensional front view showing examples of containment chambers which may be placed inside the chamber shown in FIGS. 1-3 to store objects to be sterilized and/or deimmunized therein;

FIG. 11 is a schematic block diagram showing the primary components of another embodiment of the system for sterilizing and/or deimmunizing an object;

FIG. 12 shows in further detail one example of the pump which may be utilized by the solvent delivery subsystem shown in FIG. 11;

FIG. 13 is a three-dimensional view showing another example of a pump which may be utilized by the solvent delivery subsystem shown in FIG. 11;

FIG. 14 is a three-dimensional view showing an example of a prototype of the solvent delivery subsystem shown in FIGS. 11 and 13 applying a directed volume of the non-toxic aqueous solvent to an object having infectious agents and/or immunological agents to be sterilized and/or deimmunized;

FIG. 15 shows in further detail the solvent delivery subsystem shown in FIG. 14;

FIG. 16 is a three-dimensional view showing in further detail one example of the object shown in FIGS. 14 and 15;

FIG. 17 is a three-dimensional view showing an example of a duodenoscope which may be sterilized and/or deimmunized by the system shown in FIGS. 11-16;

FIG. 18 is a three-dimensional view showing an example of an endoscope which may be sterilized and/or deimmunized by the system shown in FIGS. 11-16;

FIG. 19 is a three-dimensional view showing an example of a colonoscope which may be sterilized and/or deimmunized by the system shown in FIGS. 11-16;

FIG. 20 is a three-dimensional view showing one example of a heater which may be used by the temperature control subsystem shown in FIG. 11;

FIG. 21 is a three-dimensional view showing one example of a cooling subsystem which may be utilized by the temperature control subsystem shown in FIG. 11;

FIG. 22 is a schematic diagram showing an example of proteolysis utilized by the system shown in one or more of FIGS. 11-21;

FIG. 23 is a flowchart showing the primary steps executed by the controller subsystem shown in FIG. 11;

FIG. 24 is a block diagram showing the primary steps of one embodiment of the method for sterilizing and/or deimmunizing an object of this invention; and

FIG. 25 is a three-dimensional view showing one example of a positioning and/or storage device which may be utilized by the system and method shown in one or more of FIGS. 11-23 inside the chamber shown in FIG. 11.

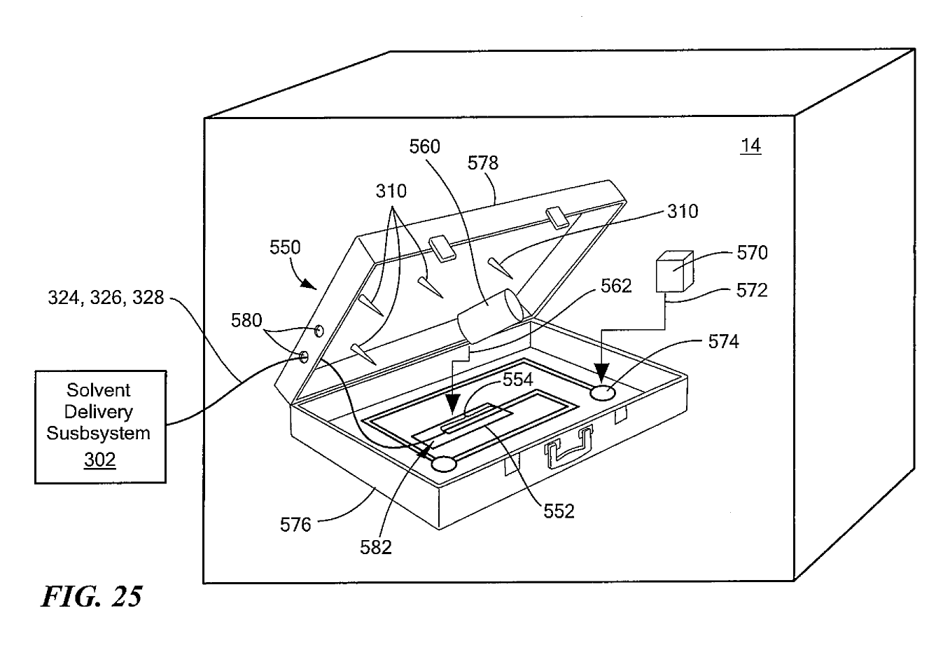

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

As discussed in the Background section above, conventional systems and methods which utilize one or more of microwaves, water, steam, and heat to sterilize and/or disinfect objects or medical equipment are complicated cumbersome systems which are pressurized, heat the water before it is applied, require a container inside a chamber to place the material to be sterilized or disinfected therein, use water to extinguish any fires that may result from using microwaves for decontamination and/or sterilization, or rely on rely on a tumbling drum. Such systems fail to teach irreversibly destroying proteins to sterilize and/or deimmunize an object.

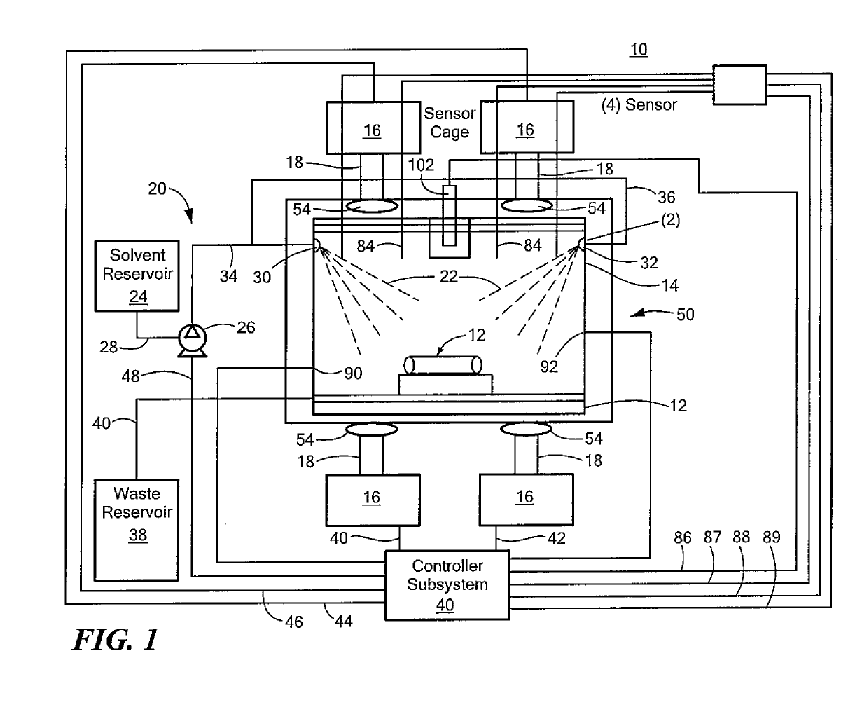

There is shown in FIG. 1, one embodiment of system 10 for sterilizing and/or deimmunizing object 12 that has been exposed to and has infectious and/or immunogenic agents thereon including, inter alia, infectious proteins, including priors and similar type infectious proteins, viruses, spore forming bacteria, vegetative bacteria, funguses, mix of bacteria protected by a biofilm, infectious or immunogenic proteins and, toxic proteins. As defined herein, object 12 to be sterilized and/or deimmunized may include medical equipment, and surgical equipment, medical devices, surgical instruments, dental equipment, devices, and instruments, veterinary equipment, devices, and instruments, or any object or thing that needs to be sterilized and/or deimmunized. System includes stationary chamber 14 at ambient pressure and configured to store object 12 to be sterilized and/or deimmunized therein. System 10 also includes an electromagnetic device coupled to the chamber to direct microwaves at the medical equipment. In one example, the electromagnetic device may include four magnetrons 16 each preferably with waveguide 18 coupled to chamber 14 as shown. In other examples, there may be more or less than four magnetrons 16 each with an associated waveguide 18. Preferably, the length of the microwaves provided by magnetron 16 with waveguide 18 is centered about a predetermined range of microwaves frequencies, e.g., between about 900 MHz and about 30 GHz or at a centered at a desired frequency. In one example, the power of magnetron 16 can be controlled, e.g., set at a desired power level, such that each magnetron 16 with waveguide 18 generates microwaves a frequency centered at about 2.45 GHz. In other examples, the power of magnetrons 16 can be set such that the frequency of the microwaves may be centered higher or lower than 2.45 GHz.

System 10 also includes solvent spray subsystem 20 coupled to chamber 14 configured to apply solvent 22 to object 12 to be sterilized and/or deimmunized such that object 12 is completely coated and/or saturated with solvent 22. In one example, solvent 22 may be at ambient temperature. In other designs, solvent 22 may be heated or cooled to improve sterilization and/or deimmunization as needed. In one example, solvent spray subsystem 20 includes solvent reservoir 24 which stores solvent 22 and pump 26 coupled to solvent reservoir 22 by line 28. Pump 26 delivers solvent 22 by line 34 to solvent atomizer 30 and/or by line 36 to solvent atomizer 32. Solvent spray subsystem 22 may also include waste reservoir 38 coupled to chamber 14 by line 40 which recovers solvent 22 directed at object 12 to be sterilized and/or deimmunized. Solvent 22 may be water, an ionic detergent and/or a non-ionic detergent or a combination thereof, e.g., Sodium dodecyl sulfate (SDS, also called sodium lauryl sulfate), Tween (Polysorbate), Triton X-100 (a nonionic surfactant that has a hydrophilic polyethylene oxide chain and an aromatic hydrocarbon lipophilic or hydrophobic group), NP-40 (nonyl phenoxypolyethoxylethanol), octyl glucoside, non-detergent sulfobetaines, mild acids and bases, hydrogen peroxide, biostatic, antimicrobial, and fungicide elements including copper, nickel, iodine, zinc, silver, gold, tin and lead. When solvent 22 is an ionic detergent or non-ionic detergent, it preferably supports denaturation of the contaminating agents or proteins such that they are more susceptible to sterilization and/or deimmunization by system 10.

System 10 also includes controller subsystem 40 coupled to the electromagnetic device comprised of one or more magnetrons 16 with waveguide 18 by lines 40, 42, 44 and 46 and solvent spray subsystem 20 by line 48. Controller subsystem 40 is configured to provide a cycle of activating solvent spray subsystem 20 for a predetermined amount of time, activating the electromagnetic device for a predetermined amount of time, and repeating the cycle a predetermined number of times to irreversibly destroy proteins on object 12 to effectively sterilize and/or deimmunize object 12. As used herein, the proteins irreversibly destroyed may be isolated proteins and/or proteins within tissue, a biomass, or an organism. The proteins on object 12 are components of infectious and/or immunogenic agents including, inter alfa, spore forming bacteria, vegetative bacteria, viruses, funguses, mix of bacteria protected by a biofilm, infectious or immunogenic proteins, and toxic proteins. In one example, solvent spray subsystem 20 is activated for 2 minutes to completely saturate or coat object 12 with solvent 22. In this example, solvent 22 is water. The electromagnetic device is then activated for 4 minutes at 1,000 watts to provide microwaves at a frequency of about 2.450 GHz. The cycle is then repeated 12 times to completely and irreversibly destroy proteins on object 12 to sterilize and/or deimmunize object 12. In one example, the microwaves generated by the electromagnetic device 16 increased the temperature inside chamber 14 to a predetermined range of temperatures, e.g. from about 20° C. to about 140° C., and to a desired temperature, e.g., about 100° C. In other examples, the amount of time the solvent is applied may be more or less than 2 minutes and the amount of time the electromagnetic device is activated may be more or less than 4 minutes to efficiently sterilize and/or deimmunize object 12. The number of cycles of activating solvent spray subsystem 20 and the electromagnetic device may be more or less than 12 cycles, e.g., 1 cycle, 4 cycles, 8 cycles, 12 cycles, 16 cycles, or any number of desired cycles to effectively sterilize and/or deimmunize object 12.

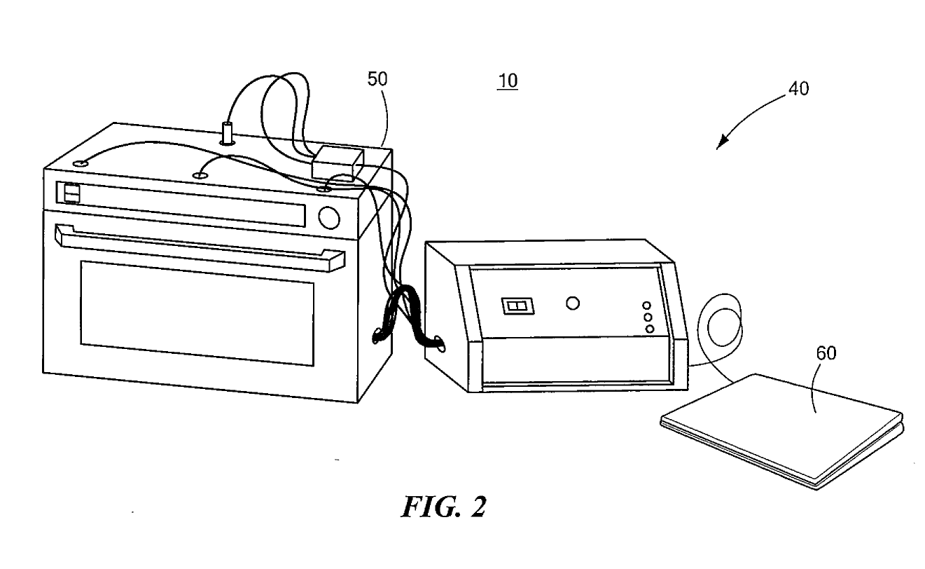

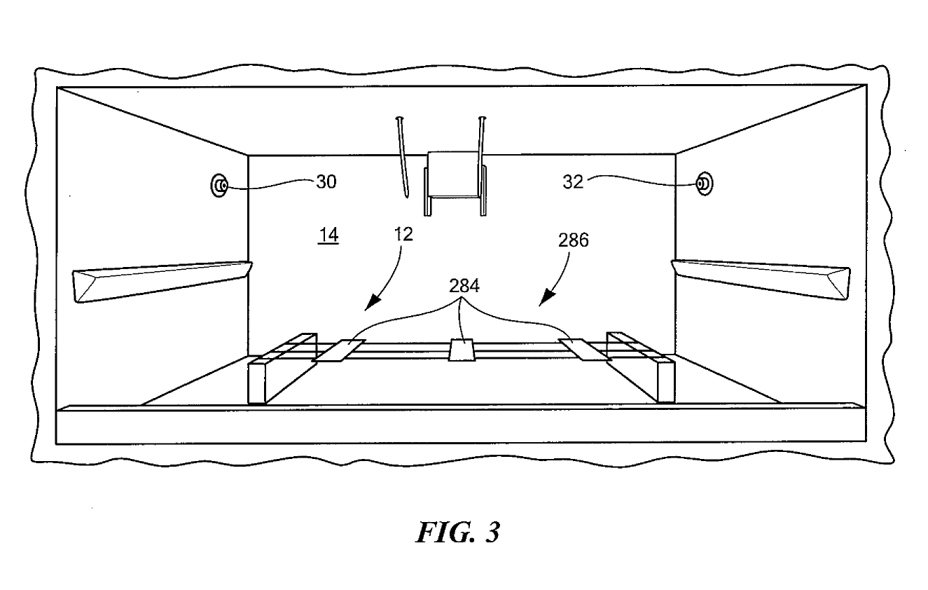

In one example, system 10 may be configured as modified microwave oven 50 as shown. In one example, modified microwave oven 50 may be a microwave oven (available from Microwave Research & Applications, Inc., Carol Stream, Ill. 60188), which has been modified as shown in FIG. 1. FIG. 2, where like parts have been given like numbers, shows three-dimensional view of system 10 configured as modified microwave oven 50 with controller subsystem 40 which preferably includes computer subsystem 60, e.g., a general purpose computer, a laptop, personal computer, or similar type computing device. FIG. 3 shows a view of the inside of modified microwave oven 50 and shows in further detail examples of atomizers 30 and 32 of solvent spray subsystem 20, FIG. 1.

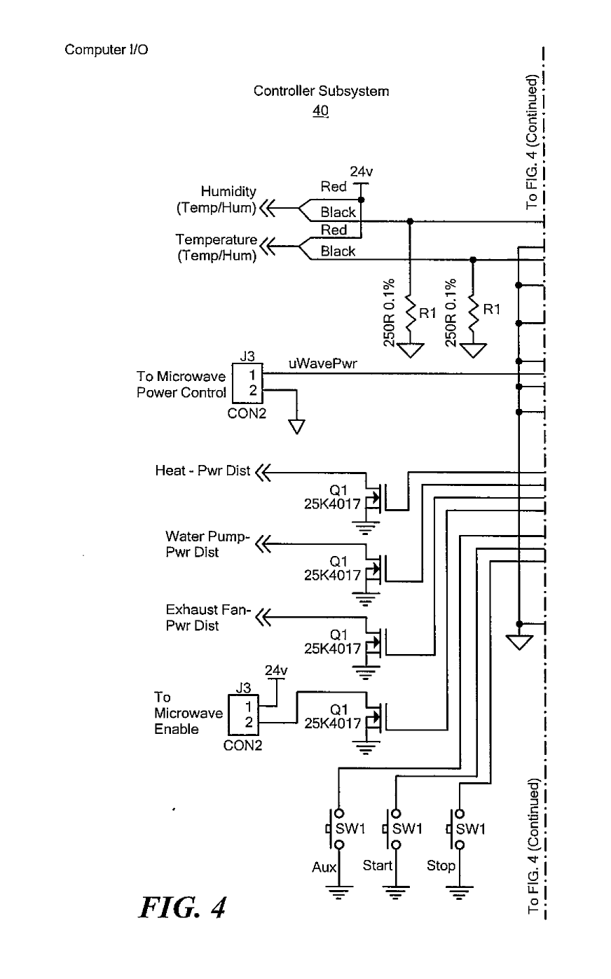

Controller subsystem 40, FIGS. 1 and 2, may include one or more processors, ASIC, firmware, hardware, andlor software (including firmware, resident software, micro code, and the like) or a combination of both hardware and software which may be part of controller subsystem 40. FIG. 4 shows a schematic circuit diagram showing in further detail the primary components of controller subsystem 40 which, in this example, includes microprocessor 100, laptop computer 60, and the associated connections to the temperature sensors 84, FIG. 1 (discussed below), microwave control, humidity sensor 102, and the like as shown.

Any combination of computer-readable media or memory may be utilized for controller subsystem 40. The computer-readable media or memory may be a computer-readable signal medium or a computer-readable storage medium. A computer-readable storage medium or memory may be electronic, magnetic, optical, electromagnetic, infrared, or semiconductor system, apparatus, or device, or any suitable combination thereof. Other examples may include an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing.

Computer program code for the one or more programs for carrying out the instructions or operation of one or more embodiments of controller subsystem 40 may be performed in an appropriate IDE, such as LabView® or similar IDE or may be written in any combination of one or more programming languages, including an object oriented programming language, e.g., C++, Smalltalk, Java, and the like, and conventional procedural programming languages, such as the “C” programming language or similar programming languages.

These computer program instructions may be provided to a processor of a general purpose computer, a controller, processor, or similar device included as part of controller subsystem 40, or separate from controller subsystem 40, or other programmable data processing apparatus to produce a machine, such that the instructions, which execute via the processor of the computer or other programmable data processing apparatus, create means for implementing the functions/acts specified in the flowchart and/or block diagram block or blocks.

The computer program instructions may also be stored in a computer-readable medium that can direct a computer, other programmable data processing apparatus, or other devices to function in a particular manner, such that the instructions stored in the computer-readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagram block or blocks.

Preferably, controller subsystem 40 controls the amount of power provided by the one or more magnetrons 16 of the electromagnetic device, a microwave output period, a duty cycle, and a mode for applying the microwaves. The microwave output period and the duty time determines the amount of time the electromagnetic device is ON and OFF. The duty cycle works in conjunction with the period. During a period, magnetrons 16, FIG. 1, are is ON for the On time, then OFF for the Off time. This repeats for the duration of the sterilization and/or deimmunization process of system 10 and the method thereof. The duty cycle identifies the percentage of time the microwave is ON.

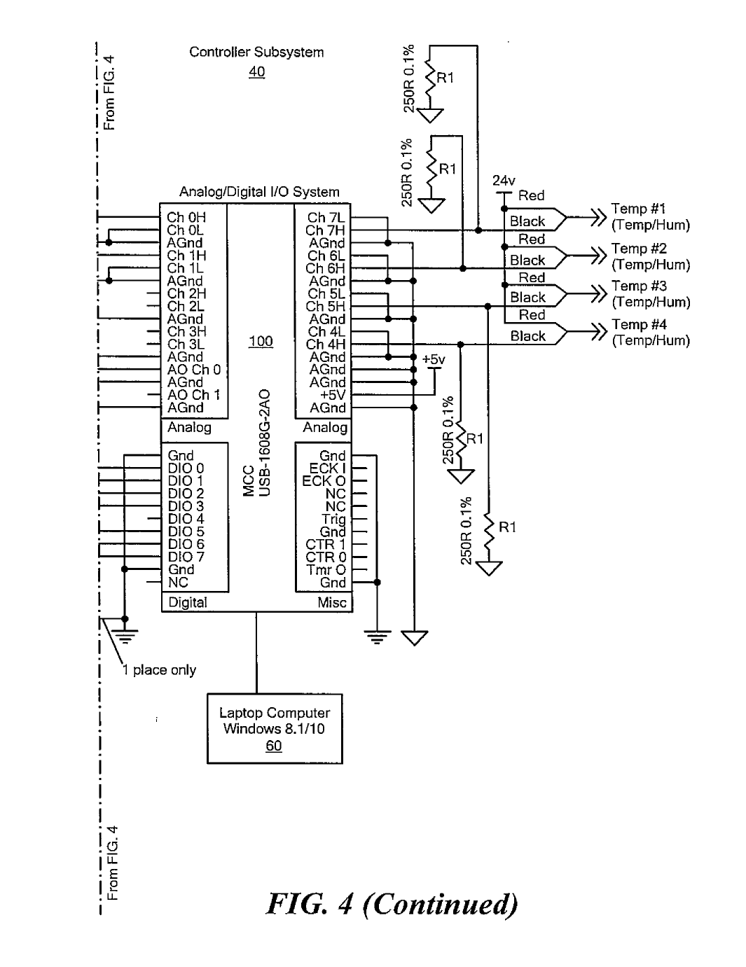

For example, FIG. 5 shows one embodiment of control interface screen 58 generated by computer subsystem 60, FIG. 2, of controller subsystem 40, FIGS. 1 and 2. In this example, control interface screen 58, FIG. 5, allows a user to set the power provided to one or more magnetrons 16, FIG. 1, and the resulting frequency of the microwaves using control buttons 62, the microwave output period using control button 64, the duty cycle using control buttons 66, and the mode using control buttons 70. In one example, the mode may include pulse width modulation (PWM) or proportional integral derivative (PID).

In one example, to provide the cycle of activating the activating solvent spray subsystem 20, FIG. 1, for a predetermined amount of time, the duty cycle is programmed (0% to 100%) using duty cycle control buttons 164 and the output period is set, e.g., to 60 seconds, using output period controls 162. The mode is set to PWM using mode control 166.

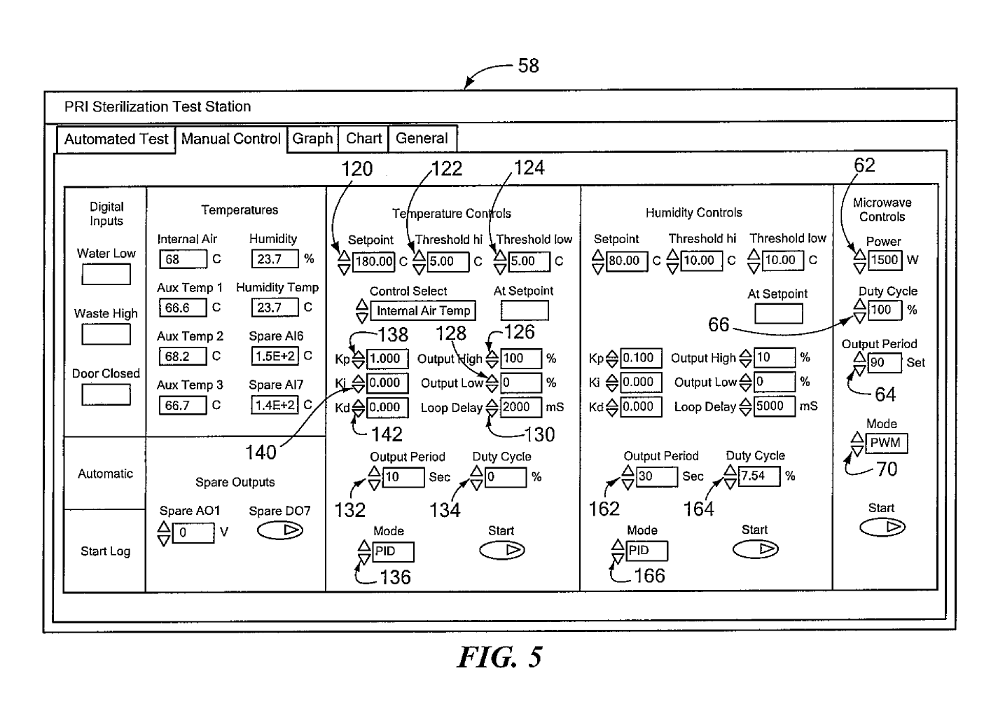

The method for sterilizing and/or deimmunizing an object of one embodiment of this invention includes providing a stationary chamber at ambient pressure configured to store an object to be sterilized and/or deimmunized therein, step 200, FIG. 6, directing the microwaves at the object inside the chamber, step 202, and applying solvent to the object to be sterilized and/or deimmunized to completely saturate and/or coat the object with the solvent, step 204. The method also includes providing a cycle of applying the solvent for a predetermined amount of time, directing the microwaves at the object for a predetermined amount of time, and repeating the cycle a predetermined number of times to irreversibly destroy proteins on the object to sterilize and/or deimmunize the object, step 206.

The following example is meant to illustrate and not limit the present invention.

### EXAMPLE 1

### Destruction of Proteins

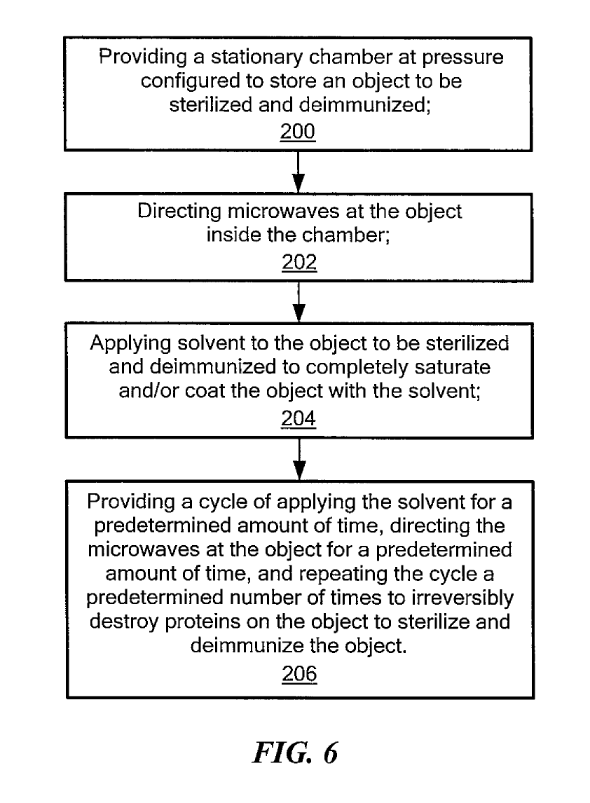

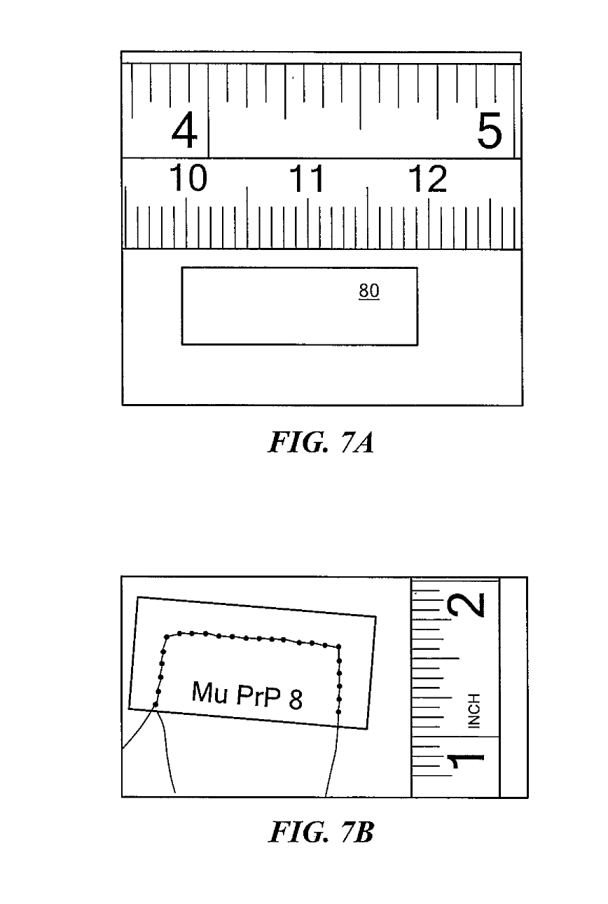

Experiments were conducted to demonstrate that a combination of vaporizing solvent, electromagnetic radiation, e.g., microwaves, and the heat generated by the microwaves which heats the environment inside the chamber and the object to be sterilized and/or deimmunized to a predetermined range of temperature including a desired temperature, irreversibly destroyed proteins to effectively sterilize and/or deimmunize an object having an infectious and/or immunogenic agent thereon. In this example, a stable PrP protein was selected for the experiments as it cannot be irreversibly destroyed using a standard autoclave. For the experiments, filter paper was cut into a strip, e.g., strip 80, FIG. 7A, and samples were created that each contained about 1 ug of a structurally robust mouse PrP protein and wrapped in 100% cotton paper and sewed in place as shown in FIG. 7B to avoid extraneous contamination. This containment was placed in a second layer of 100% cotton paper to increase stability during treatment. The samples were treated with system 10 and the method thereof as discussed above for differing number cycles of applying moisture saturation and microwaves. After treatment, the samples were subjected to standard Western blot analysis. For this, the samples were suspended in loading buffer, boiled to denature the proteins and run on a denaturing protein gel to separate intact protein and substantially intact proteins from small polypeptides and amino acids. The samples were then transferred to a nylon membrane. The membranes were then incubated with a primary antibody that specifically binds to a region near the C-terminus of the protein and visualizes with a secondary HRP-labelled antibody. The process enables the detection of any intact or partially intact protein sample. Proteins irreversibly broken into small polypeptides and amino acids and destroyed will not be visualized. With the Western Blot analysis, it was possible to see that certain combinations of the treatment cycles of system 10 and the method thereof as discussed above with reference to one or more of FIGS. 1-6 irreversibly and completely destroyed the protein samples, shown by the absence of protein bands of gel as shown by lane 5, indicated at 82, FIG. 8. Other treatment conditions did not destroy the highly robust proteins.

### EXAMPLE 2

### Additional Parameter Testing

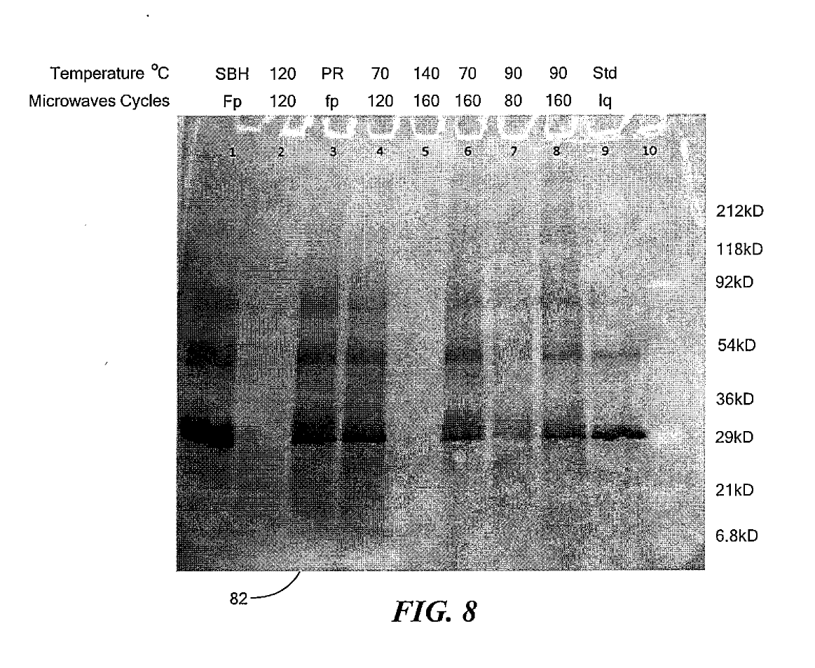

Testing was conducted to determine the ability of one or more embodiments of system 10 and the method thereof to sterilize and/or deimmunize object 12 material using biologic indicator strips impregnated with a set number of biologic spores. For the procedure, biologic indicator strips 284, having infectious agent thereon, FIG. 9, were placed in chamber 14, FIGS. 1 and 3, e.g., indicated at 286, FIG. 3, and subjected to a predetermined number of cycles of applying solvent and microwaves using system 10 and the method thereof as discussed above. After the sterilization cycles, biologic indicator strip 286 were placed in culture media and incubated for 10 days. If bacteria grow during that period, sterilization failed. Only if no bacteria grew during the 10 day culture system 10 and the method thereof be qualified for sterilization.

Depending on the sterilization technology being used, one of three spore forming bacteria is used to determine successful sterilization. To qualify steam sterilization (autoclave), the bacterial species G. stearothenuophilus (10⁴-10⁶ spores) was used. For Gamma radiation sterilization, the bacterial species B. pumilus (10⁴-10⁶ spores) was used. For Ethylene Oxide (ETO) sterilization, the bacterial species B. atrophaeus (10⁴-10⁶ spores) was used. B. atrophaeus is the standard surrogate species for anthrax (pathogenic B. anthracis).

For the experiments using standard biologic indicator strips 284, FIG. 9, the temperature of chamber 14 and object 12 was varied, e.g., about 60° C. to about 160° C. The saturation of object 12 and the power and cycle of the electromagnetic device were applied as discussed above with reference to one or more of FIGS. 1-6. System 10 and the method thereof was able to sterilize G. stearothermophilus and B. pumilus spores at between about 100° C. and about 120° C. System 10 and the method thereof was able to sterilize B. atrophaeus at between 120° C. and 140° C.

Additional testing was conducted using the bacterial species B. atrophaeus (anthrax surrogate). For this experiment, the number of spores inserted was logarithmically increased from 10⁴-10⁶ spores to the extremely high number of 10⁻¹⁰ spores. The test was run at 150° C. and 80 cycles. System 10 and the method thereof was able to successfully kill all the spores.

The result is system 10 and the method thereof completely and irreversibly destroys proteins on an object to efficiently and effectively sterilize and/or deimmunize any object that needs to be sterilized and/or deimmunized. System 10 effectively irreversibly destroys proteins that are components of infectious and/or immunogenic agents including spore forming bacteria, vegetative bacteria, viruses, funguses, mix of bacteria protected by a biofilm, infectious or immunogenic proteins, and toxic proteins that may be found on an object to be sterilized and/or deimmunized. System 10 is easy to use, does not need to be pressurized, and does not require using a container inside the chamber. System 10 and the method thereof is also much less complex than the conventional systems discussed in the Background section above. The proteins irreversibly destroyed by system 10 includes prions which are a unique category of transmissible infectious agents that cause a wide range of diseases.

In one embodiment, system 10, FIG. 1, may include mode stirrer 54 coupled between waveguide 18 and chamber 14 as shown to provide a more uniform distribution of the microwaves generated by magnetron 16 and waveguide 18.

System 10, FIG. 1, may also include a plurality of temperature sensors 84 coupled to controller subsystem 40 by lines 86, 87, 88 and 89 as shown configured to measure the temperature inside chamber 14.

In one design, instead of utilizing the electromagnetic device discussed above with reference to FIG. 1 to heat inside the environment inside chamber 14 and object 12 to be sterilized and/or deimmunized to the predetermined range of temperatures, e.g., 20° C. to 140° C., e.g., about 100° C., system 10 may also include one or more heaters, e.g., heaters 90, 92 coupled to the walls of chamber 14 as shown to heat the environment inside chamber 14 and object 12 to be sterilized and/or deimmunized to the predetermined range of temperatures including a desired temperature.

In one design, controller subsystem 40 may include temperature set point controls 120, FIG. 5, threshold high controls 122, threshold low controls 124, output high controls 126, output low controls 128, loop delay controls 130, output period controls 132, duty cycle controls 134, and mode controls 136, Kp (proportional coefficient) controls 138, Ki (integral coefficient) controls 140, and Kd (derivative coefficient) controls 142, of which one or more may be utilized to set the temperature parameters provided by heaters 90, 92, FIG. 1 to heat the environment inside chamber 14 and object 12 to the predetermined range of temperatures or a desired temperature

In one example, controller subsystem 40 may be configured to provide a cycle of activating solvent spray subsystem 20 for a predetermined amount of time, activating heating devices 90, 92 for a predetermined amount of time, and activating the electromagnetic device a predetermined amount of time, and repeating the cycle a predetermined amount of times to irreversibly destroy proteins on object 12 to sterilize and/or deimmunize object 12.

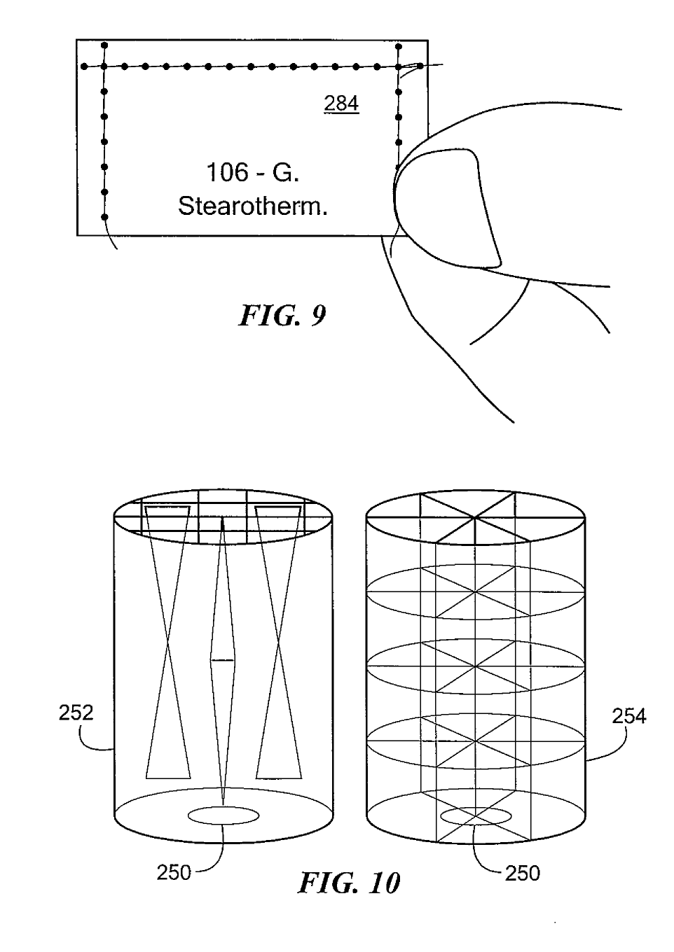

In one design, system 10 may include rotating cog 250, FIG. 10, which is preferably coupled to the floor of chamber 14 shown in FIGS. 1 and 3. In this design, system 10 also includes a containment chamber 252 or (containment chamber) 254 coupled to cog 250. Containment chamber 252 is designed to store larger medical equipment to be sterilized and or deimmunized and containment chamber 254 is designed to store smaller medical equipment to be sterilized and/or deimmunized as shown.

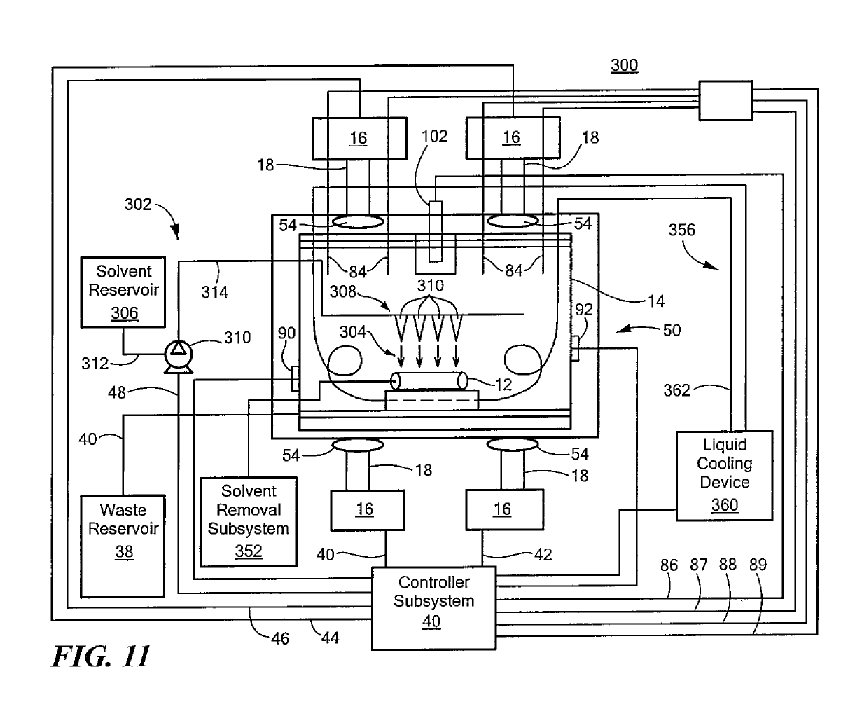

System 300, FIG. 11, where like parts have been given like numbers, for sterilizing and/or deimmunizing an object of another embodiment of this invention includes stationary chamber 14 at ambient pressure configured to store object 12 to be sterilized and/or deimmunized therein, similar as discussed above with reference to one or more of FIGS. 1-10.

System 10 also includes solvent delivery system 302 coupled to chamber 14 configured to apply a directed volume of a non-toxic aqueous solvent 304 to coat and wet object 12 to optimally hydrate proteins of infectious agents and/or immunological agents in or on object 12 for proteolysis of the proteins of the infectious agents and/or immunological agents in or on object 12. As known by those skilled in the art, proteolysis is the breakdown of proteins into smaller peptides or amino acids. In one design, non-toxic aqueous solvent 304 may be water, an alcohol solution, e.g., 70% isopropyl, 60% ethyl alcohol, or other similar type alcohol solution, hydrogen peroxide, an ionic detergent, and/or a non-ionic detergent, or any combination thereof. Details associated with non-toxic aqueous solvent 304 being an ionic detergent, and/or a non-ionic detergent are discussed in detail above with reference to FIG. 1. In one deign, non-toxic aqueous solvent 304 is stored in solvent reservoir 306.

In one design, solvent delivery subsystem 302 includes solvent delivery device 308 which includes one or more ports 310 which apply non-toxic aqueous solvent 304 to or on object 12 to coat and wet objet 12 to optimally hydrate the proteins of the infectious agents and/or immunological agents in or on object 12 for proteolysis.

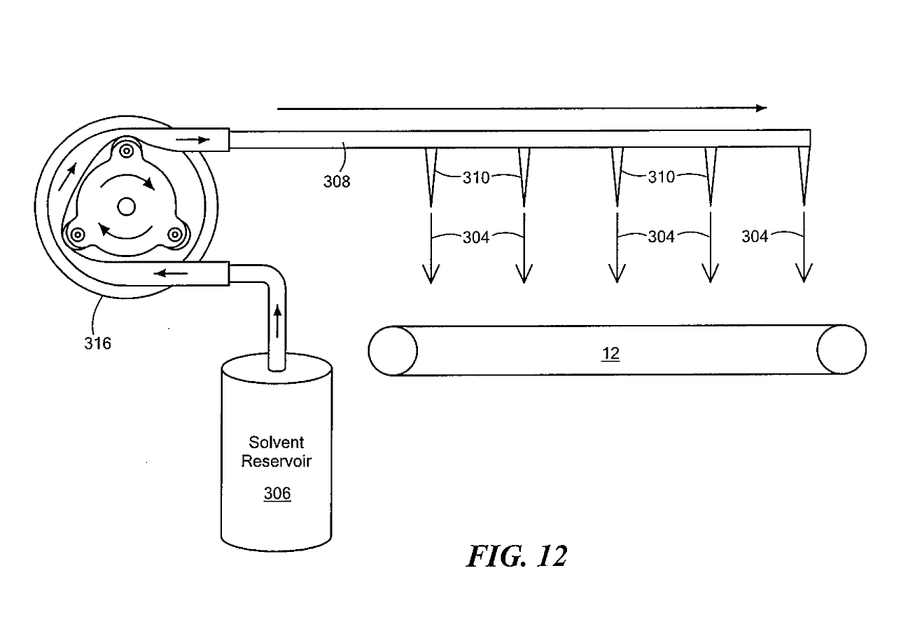

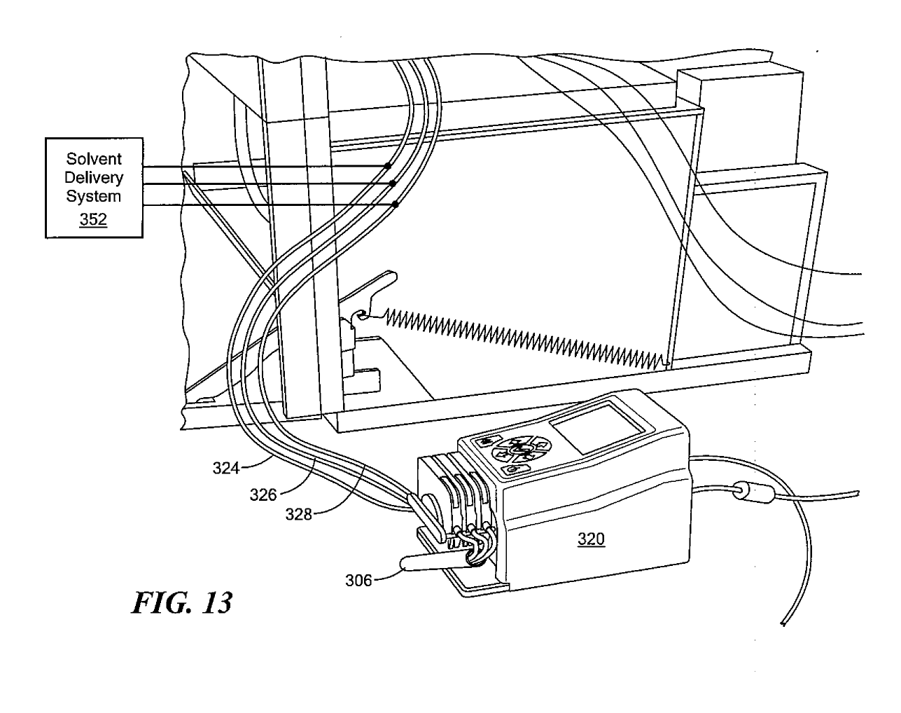

Solvent delivery subsystem 302 also includes pump 310 coupled to solvent reservoir 306 by line 312 and to solvent delivery device 308 by line 314. In one design, pump 310 may be a peristaltic pump, e.g., peristaltic pump 316, FIG. 12, where like parts have been given like numbers, which is coupled to solvent delivery device 308 with ports 310. Another example, pump 310, FIG. 11, may be configured as a peristaltic pump 320, FIG. 13, e.g., an Ismatec 15M4312, available from Cole-Parmer GmbH, Wertheim, Germany. In one design, peristaltic pump 320 may be coupled to solvent delivery device 308, FIGS. 11 and 12, to apply the directed volume of non-toxic aqueous solvent 304 to coat and wet object 12 to optimally hydrate the proteins of the infectious agents and/or immunological agents in or on object 12 for proteolysis.

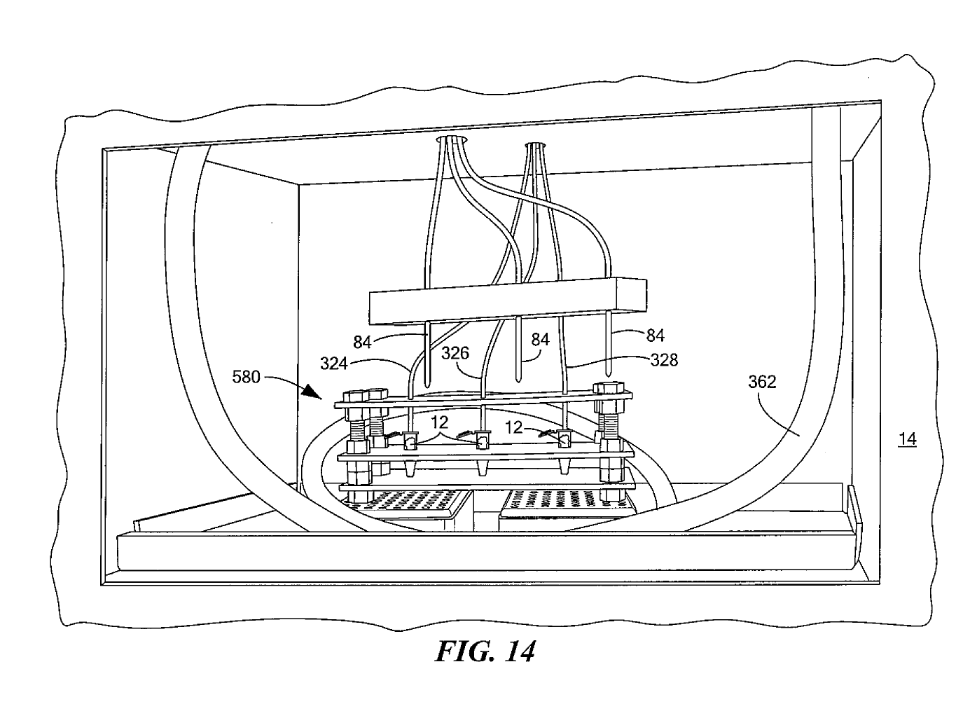

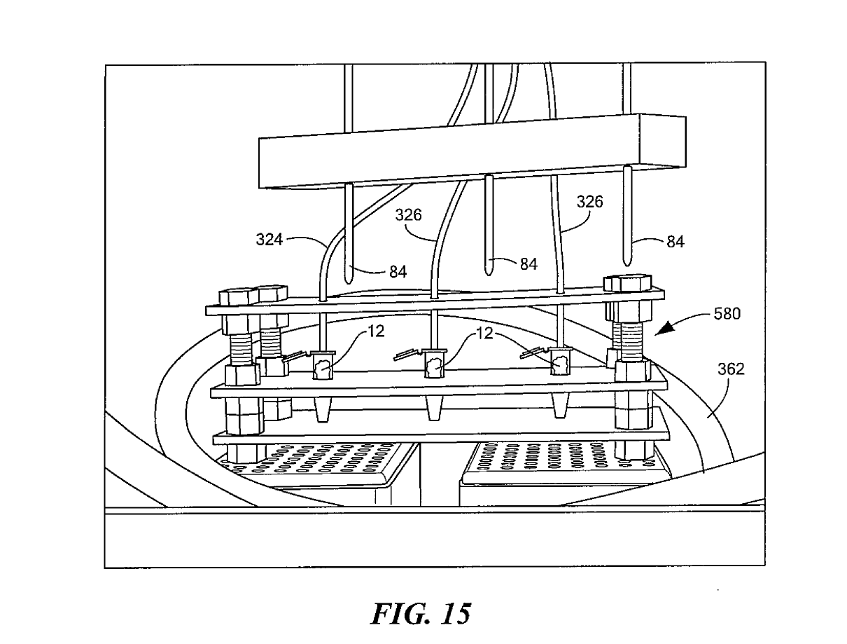

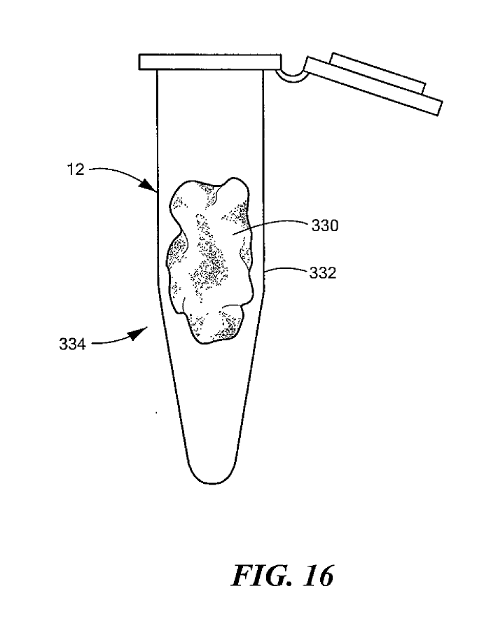

In one prototype design of system 300, FIG. 11, and the method thereof, solvent delivery subsystem 302 may include one or more fluid lines, e.g., one or more fluid lines 324, 326, and/or 328, FIG. 13, coupled to peristaltic pump 320 which preferably enter chamber 14 as shown in FIGS. 14 and 15 and apply a directed volume of non-toxic aqueous solvent 304 to object 12. In this prototype testing embodiment of system 300, FIG. 11, object 12, FIGS. 14 and 15 preferably includes a small sample of proteins of infectious agents and/or immunological agents disposed on small cores of a sponge material suspended in a modified polypropylene tubes, e.g., sponge material 330, FIG. 16, having a small sample of proteins of infectious agents and/or immunological agents suspended therein and placed inside modified polypropylene tube 332 having its end removed, as indicated at 334, as discussed in detail in Example 3 below. In this example, the directed volume of a non-toxic aqueous solvent 304 to coat and wet object 12 to optimally hydrate proteins of infectious agents and/or immunological agents in or on object 12 for proteolysis about 25 to 50 ul. of non-toxic aqueous solvent 304 provided by solvent delivery subsystem 302 about every 10 to 15 minutes to coat and rewet sample 12, as discussed in detail in Example 3 below.

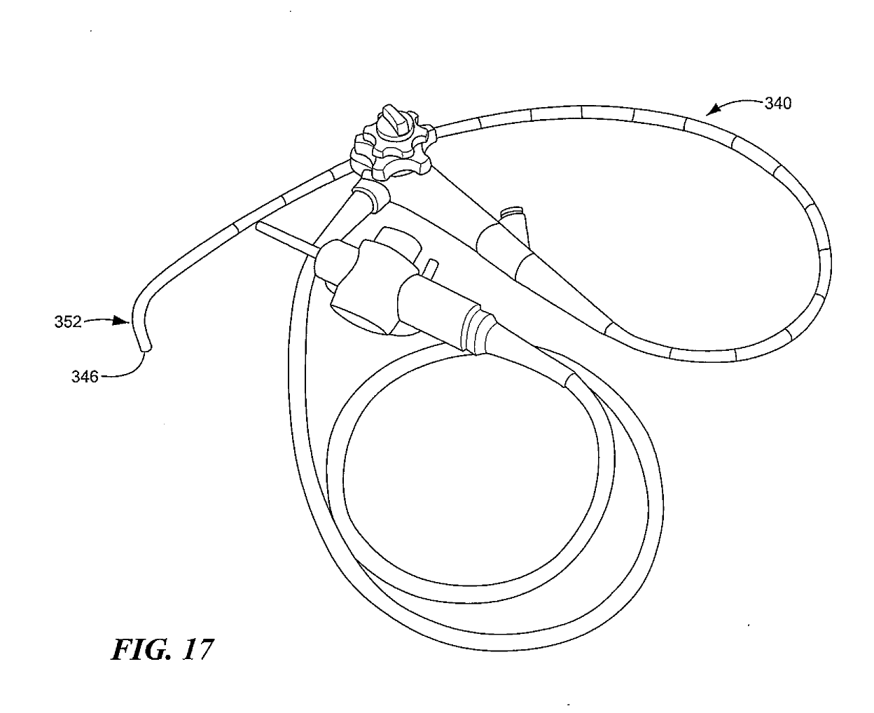

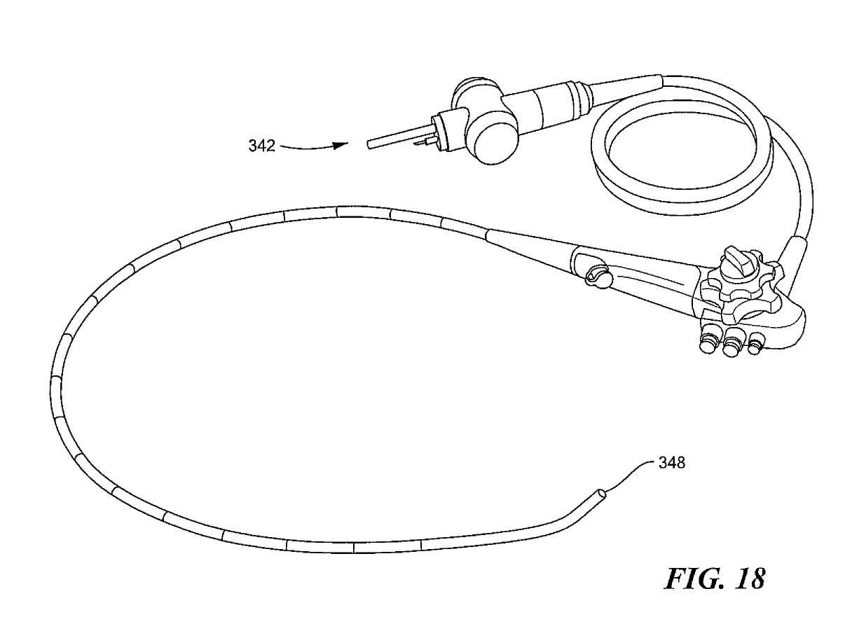

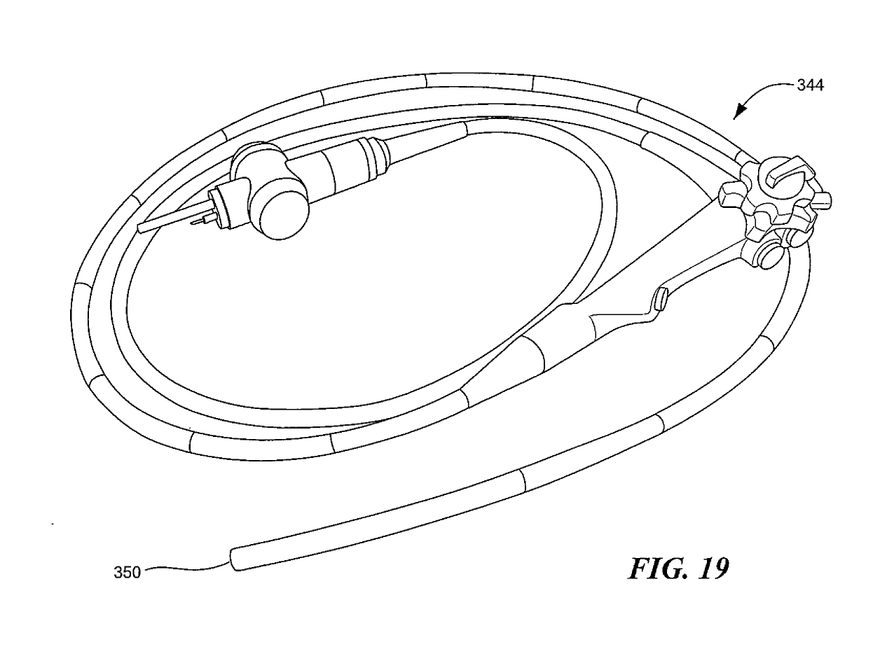

In other examples, object 12 may be to be sterilized and/or deimmunized by system 300 and the method thereof may include medical equipment, and surgical equipment, medical devices, surgical instruments, dental equipment, devices, and instruments, veterinary equipment, devices, and instruments, or any object or thing that needs to be sterilized and/or deimmunized. In one example, the surgical equipment may include a gastrointestinal scope, e.g., a duodenoscope 340, FIG. 17, endoscope 342, FIG. 18, colonoscope 344, FIG. 19, or any similar type gastrointestinal scope. In one example, object 12 may include a lumen in the duodenoscope, the endoscope, or the colonoscope, e.g., lumen 346, FIG. 17 of duodenoscope 340, lumen 348, FIG. 18 of endoscope 342, lumen 350, FIG. 19, of colonoscope 344, or any similar type lumen of a gastrointestinal scope or any lumen of any medical, surgical, dental, veterinary device or any object or thing that has a lumen that needs to be sterilized and/or deimmunized. Object 12 may also include a biofilm on the lumen of any object to be sterilized and/or deimmunized, discussed above, or any similar type lumen of a gastrointestinal scope or any lumen of any medical, surgical, dental, veterinary device or any object or thing that has a lumen that needs to be sterilized and/or deimmunized. In one example, object 12 may include movable tip of the duodenoscope, e.g., movable tip 352, FIG. 17.

In one example, to sterilize and/or deimmunize object 12 configured a lumen, a directed volume of non-toxic aqueous solvent 304 solvent may be injected into the lumen by one or more of fluid lines 324, 326, and/or 326, FIG. 13 and peristaltic pump 320 or peristaltic pump 316, FIG. 12, of solvent delivery subsystem 302, FIG. 11. In this example, the directed volume of non-toxic aqueous solvent 304 is sufficient to fill the entire length of the lumen of object 12. Once the lumen is filled with non-toxic aqueous solvent 304 sufficiently to coat and wet the lumen or a biofilm on the lumen to optimally hydrate proteins of infectious agents and/or immunological agent thereon for proteolysis, the directed volume of non-toxic aqueous solvent 304 injected into the lumen is then preferably removed by solvent removal subsystem 352, FIGS. 11 and 13, e.g., a vacuum or similar type device.

System 300, FIG. 11. also includes an electromagnetic device, e.g., as discussed above with reference to FIG. 1, coupled to chamber 14 configured to direct microwaves at object 12 to induce proteolysis of the proteins of the infectious agents and/or immunological agents in or on object 12 and generate heat to dry non-toxic aqueous solvent 304 in or on object 12. In one example, each of magnetrons 16 of the electromagnetic device preferably emit microwaves at frequency of about 2.45 GHz, a wavelength of about 122 mm, and a maximum power of about 8,000 W each to provide a total available power about 3,200 W into chamber 14. Preferably, a controlled percentage of the maximum available power is utilized, e.g., about 15% to 100%, as discussed in detail in Example 3 below. In other examples, magnetron 16 of the electromagnetic device may emit microwaves at different wavelengths and frequencies and operate at different power levels.

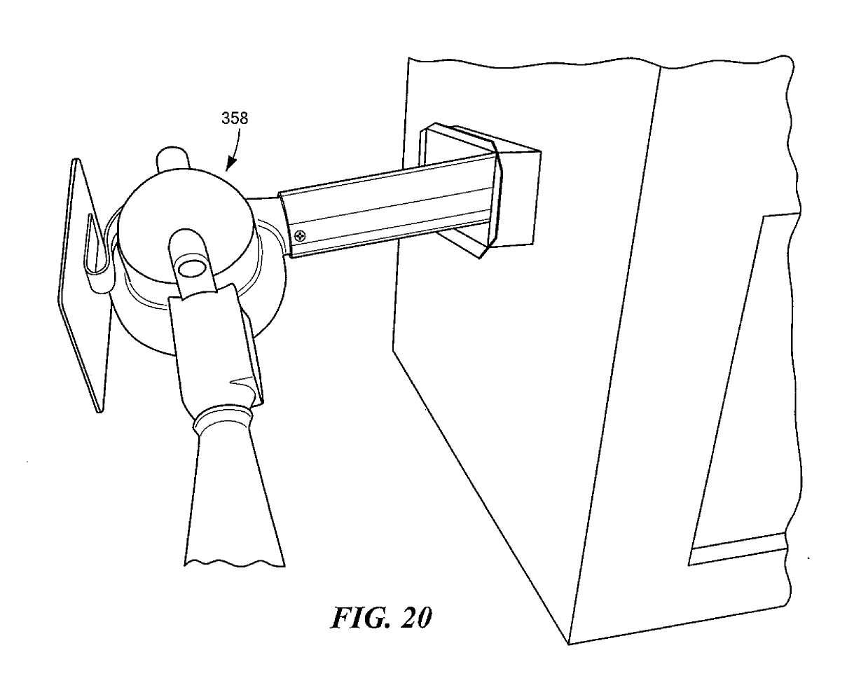

System 300, FIG. 11, also includes temperature control subsystem 356 coupled to chamber 14 configured to control the temperature of chamber 14 to induce a temperature of the proteins of the infectious agents and/or immunological agents in or on object 12 which accelerates proteolysis of the proteins and dries non-toxic aqueous solvent 304 in or on object 12. In one design, temperature control subsystem 356 may utilize the electromagnetic device discussed above to heat the chamber to induce a temperature of the proteins of the infectious agents and/or immunological agents in or on object 12 which accelerates proteolysis of the proteins and dries non-toxic aqueous solvent 304 in or on object 12. Temperature control subsystem 356 may also include one or more heating devices to direct heat into chamber 14, e.g., heaters 90, 92 coupled to the walls of chamber 14 as shown to heat the environment inside chamber 14 and object 12 and the proteins of the infectious agents and/or immunological agents in or on object 12 in chamber 14 to be sterilized and/or deimmunized. In one example, one or more of heaters 90, 92 may be a heating device, e.g., blower 358, FIG. 20, coupled to chamber 14 as shown, or similar type heating device.

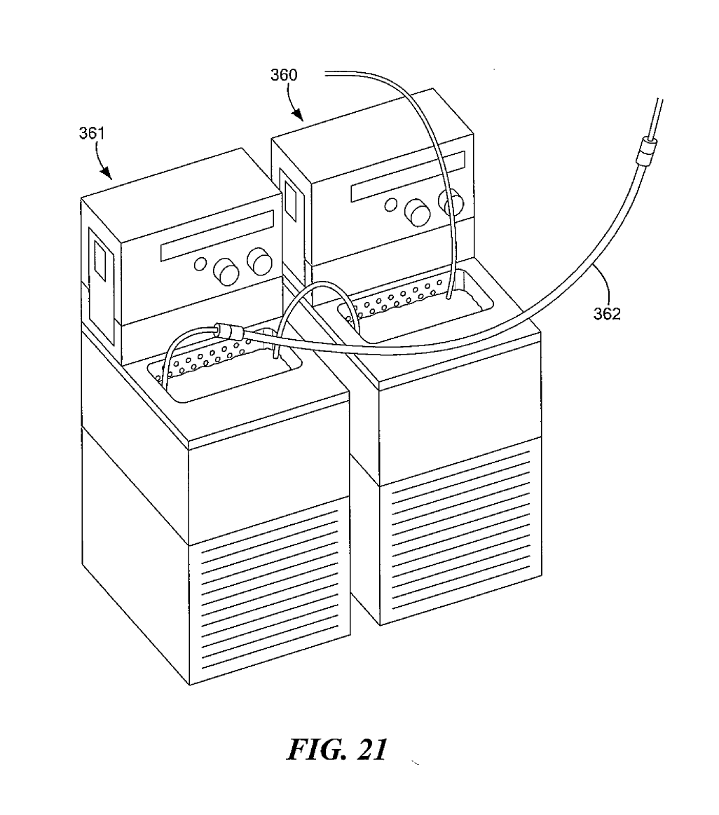

Temperature control subsystem 356, FIG. 11, may also include liquid cooling device 360 coupled to cooling line 362 which cycles a cooled liquid in cooling line 362 through chamber 14 as shown. FIG. 14 shows in further detail one example of cooling line 362 disposed in chamber 14 and wrapped as a coil in chamber 14 to cool the environment and object 12 and the proteins of the infectious agents and/or immunological agents in or on object 12 in chamber 14. FIG. 21 show one example of liquid cooling devices 360, 361 coupled to cooling line 362 used to deliver the cooled liquid in cooling line 362 to chamber 14. One or both of cooling devices 360, 361 may be utilized when cooling of chamber 14 is needed. One example of cooling devices 360, 361 may be Neslab RTE-111 systems (available from Neslab Instruments, Inc., Newington, N.H. 03801).

In one example, the temperature maintained in chamber 14 and object 12 and the proteins of the infectious agents and/or immunological agents in or on object 12 by temperature controlling subsystem 356 is in the range of about 25° C. to about 100° C., e.g., preferably about 60° C. to about 70° C. Such a temperature range prevents damage to object 12, most notably when object 12 is a gastrointestinal scope, e.g., a duodenoscope 340, FIG. 17, endoscope 342, FIG. 18, colonoscope 344, FIG. 19, or similar type gastrointestinal scope.

System 300, FIG. 11, also includes controller subsystem 40 coupled to solvent delivery subsystem 302, the electromagnetic device, and temperature control subsystem 356. Controller subsystem 40, in this embodiment for system 300, provides a wet/dry cycle which includes activating solvent delivery subsystem 302 for a predetermined amount of time to wet and hydrate the proteins for proteolysis, activating the electromagnetic device for a predetermined amount of time to induce proteolysis of the proteins and dry non-toxic aqueous solvent 304 in or on object 12, and activating temperature control subsystem 356 a predetermined amount of time to accelerate proteolysis of the proteins and dry non-toxic aqueous solvent 304 in or on object 12 and repeating the wet/dry cycle a predetermined amount of times to irreversibly destroy proteins in or on object 12 to sterilize and/or deimmunize object 12. Details of an example of the wet/dry cycle and repeating the wet/dry cycle are discussed in detail in Example 3 below.

The result is system 300 applies a controlled directed volume of a non-toxic aqueous solvent to coat and wet an object to be sterilized and/or deimmunized. The directed volume of the non-toxic aqueous solvent optimally hydrates the proteins of infectious agents and/or immunological agents in or on the object for proteolysis. The electromagnetic device induces proteolysis and the temperature control subsystem induces a temperature of the proteins which accelerates proteolysis of the proteins. The electromagnetic device and the temperature control subsystem provide a temperature in the chamber which promotes proteolysis of the proteins, dries the non-toxic aqueous solvent in or on the object, all while preventing damage to the object 12, most notably, a gastrointestinal scope such as an duodenoscope, endoscope, a colonoscope, or similar type gastrointestinal scope.

The wet/dry cycle of activating the solvent delivery subsystem for a predetermined amount of time to wet and hydrate the proteins for proteolysis, activating the electromagnetic device for a predetermined amount of time to induce proteolysis and dry the non-toxic aqueous solvent in or on the object, and activating the temperature control system a predetermined amount of time to accelerate the proteolysis of the proteins and dry the non-toxic aqueous solvent in or on the object, and repeating the wet/dry cycle a predetermined number of times irreversibly destroys the proteins in or on the object to effectively and efficiently sterilize and/or deimmunize the object.

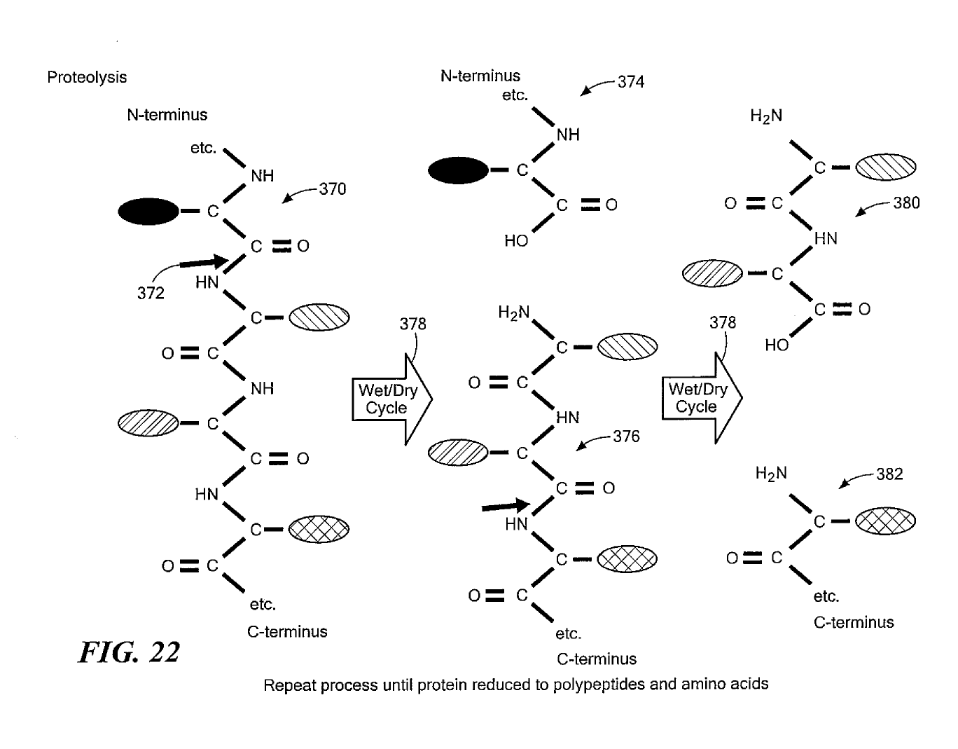

FIG. 22 shows an example of proteolysis of a protein chain where a wet/dry cycle, indicated at 368, provided system 300, shown in one or more of FIGS. 11-20, induces proteolysis of protein 370, FIG. 22, of the infectious agents and/or immunological agents in or on object 12 to break the bond between the carboxylic group and NH group, indicated at 372, thereby breaking protein 370 into polypeptide 374 and protein fragment 376. The next wet/dry cycle provided by system 300, indicated at 378, breaks protein fragment 376 into smaller protein fragment 380 and polypeptide 382. The wet/dry cycle is repeated until the proteins of any infectious agents and/or immunological agents in or on object 12 are reduced to small polypeptides and amino acids thereby irreversibly destroying all proteins in or on object 12 to effectively and efficiently sterilize and/or deimmunize the object 12. See Example 3 below.

One exemplary flowchart of the primary steps executed by controller subsystem 40, FIG. 11 of system 300 and the method thereof, to perform the wet/dry cycle and repeating the wet/dry cycle as discussed above with reference to one or more of FIGS. 11-22 includes start process, step 400, FIG. 22. The method is then selected, step 402, e.g., as shown in Table 2 below. A load method is then provided, step 404, e.g., as shown in Table 2 below. Sterilization is then initiated by operator input, step 406. The next instruction is then provided, step 408, e.g., as shown in Table 2 below. Instructions are then sent to temperature control subsystem 356, FIG. 11, solvent delivery subsystem 302, and the electromagnetic device to set the desired temperature, flow rate, and amount of energy provided by the electromagnetic device, step 410. The temperature is initiated, a PID mode is activated, and the temperature set point is provided, step 412. The temperature in chamber 12 is then read, step 414. The temperature process variable (PV) is then calculated, step 416. The temperature output is then adjusted, step 418. The cycle time of solvent delivery subsystem 302 is then determined, step 420. The solvent flow is turned on for a desired injection volume, step 422. Solvent flow is then turned off for the end of the cycle, step 424, and steps 420 to 424 are repeated as needed. The microwave cycle, power, duty, and period for the electromagnetic device is determined, step 426. The power level of the electromagnetic device is then set, step 428. The cycle for duty and period for the electromagnetic device is then determined, step 430. Steps 426 to 430 are repeated as needed. The process is then teuninated and a report is run, step 432.

Table 2 below shows exemplary various parameters utilized by controller subsystem 40 to provide the wet/dry cycles discussed above with refence to one or more of FIGS. 11-22 to irreversibly destroying all proteins in or on object 12 to effectively and efficiently sterilize and/or deimmunize the object 12.

Other parameters may be utilized by controller 40 to vary the wet/dry cycles as needed.

TABLE 2
Parameters used by the controller subsystem.

| Method Setup | Value | Notes |
|---|---|---|
| Set temp setpoint | 70 | |
| Run Temp Control | on | |
| Run Microwave control | off | Safety |
| Set Microwave Duty Cycle | 35 | |
| Set Microwave Period | 90 | |
| Set Microwave Power | 815 | |
| Wait Time Delay | 0 | |
| Run Microwave control | on | Turn On uWave |
| Wait time delay | 1200 | 20 min |
| Run Microwave control | off | uWave Off |
| Wait time delay | 60 | add fluid |
| Run Microwave control | on | Turn On uWave |
| Wait time delay | 900 | 15 min |
| Run Microwave control | off | uWave Off |
| Wait time delay | 60 | add fluid all |
| Run Microwave control | on | Turn On uWave |
| Wait time delay | 900 | 15 min |
| Run Microwave control | off | uWave Off |
| Wait time delay | 60 | add fluid |
| Run Microwave control | on | Turn On uWave |
| Wait time delay | 900 | 15 min |
| Run Microwave control | off | uWave Off |
| Wait time delay | 60 | add fluid all |
| Run Microwave control | on | Turn On uWave |
| Wait time delay | 900 | 15 min |
| Run Microwave control | off | uWave Off |
| Wait time delay | 60 | add fluid |
| Run Microwave control | on | Turn On uWave |
| Wait time delay | 900 | 15 min |
| Run Microwave control | off | uWave Off |
| Wait time delay | 60 | add fluid all |
| Run Microwave control | on | Turn On uWave |
| Wait time delay | 900 | 15 min |
| Run Microwave control | off | uWave Off |
| Wait time delay | 60 | add fluid |
| Run Microwave Control | on | |
| Wait time delay | 900 | 15 min |
| Run Microwave control | off | Complete |

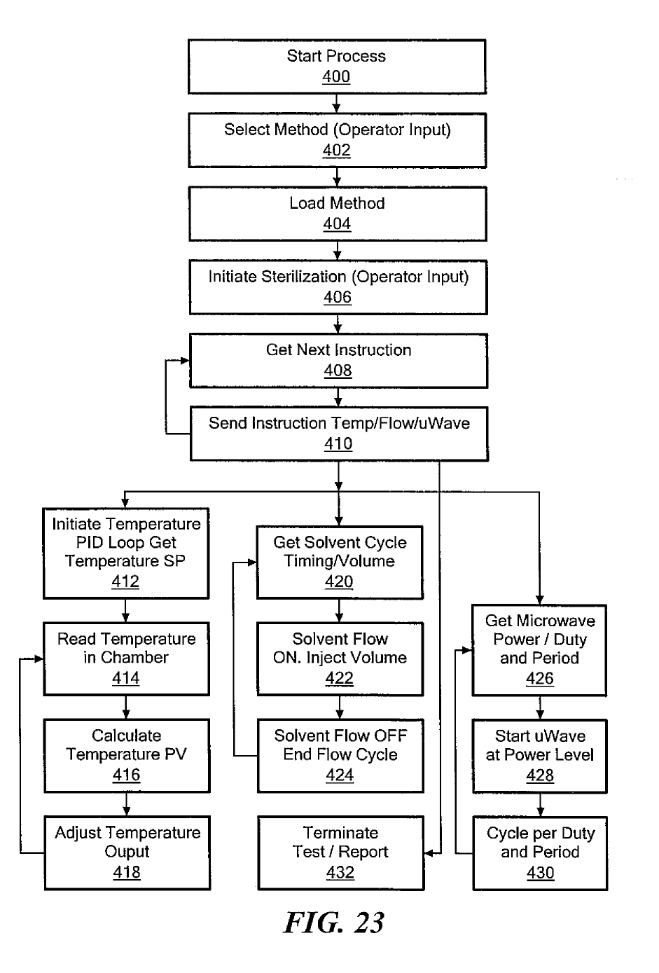

One example of the method for sterilizing and deimmunizing an object of this invention includes providing a stationary chamber at ambient pressure configured to store an object to be sterilized or deimmunized therein, step 450, FIG. 23. A directed volume of a non-toxic aqueous solvent is applied to coat and wet the object to optimally hydrate the proteins of infectious agents and/or immunological agents in or on the object for proteolysis, step 452. Microwaves are directed at the object to induce proteolysis of the proteins and generate dry heat to dry the non-toxic aqueous solvent in or on the object, step 454. The temperature of the chamber is controlled such that the temperature of the proteins in or on the objects accelerates proteolysis of the proteins in or on the object and dries the non-toxic aqueous solvent in or on the object, step 456. A wet/dry cycle is provided including coating and wetting the proteins to optimally hydrate the proteins for proteolysis, applying microwaves a predetermined amount of time to induce proteolysis of the proteins and dry the non-toxic aqueous solvent in or on the object, controlling the temperature a predetermined amount of time to accelerate proteolysis of the proteins and dry the non-toxic aqueous solvent in or on the object, and repeating the wet/dry cycle a predetermined number of times to irreversibly destroy proteins in or on the object to sterilize and/or deimmunize the object, step 458, FIG. 19.

In one embodiment, the method includes activating the electromagnetic device a predetermined amount of time to heat the chamber, the object, and the proteins of the infectious agents and/or immunological agents on the object to a predetermined range of temperatures, e.g., 60-100° C., e.g., preferably about 60° C. to 70° C., as discussed above.

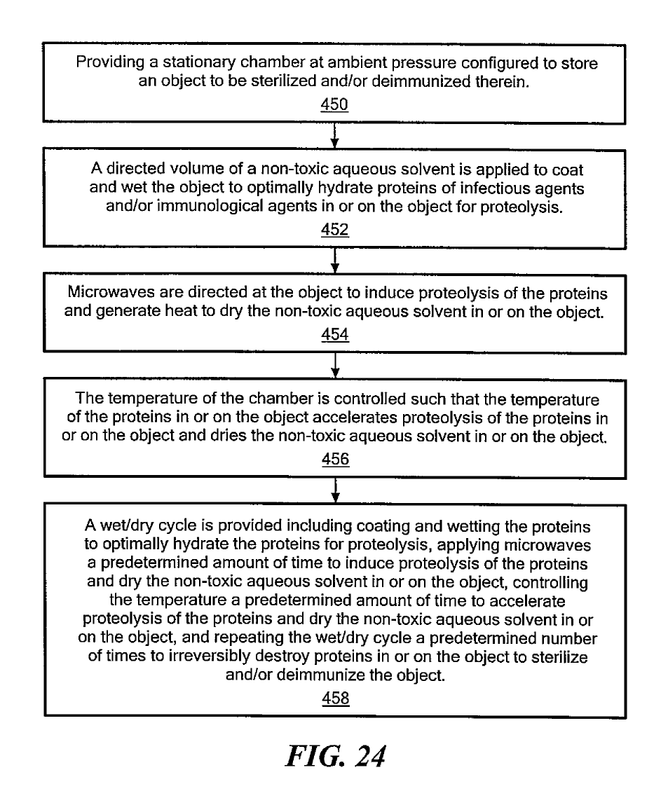

System 300 and the method thereof shown in one or more of FIGS. 11-23 may also include positioning and/or storage device 550, FIG. 24, located inside chamber 14 as shown. Positioning and/or storage device 550 is preferably configured to secure and position object 12 therein to increase proteolysis and irreversible destruction of proteins in or on object 12 to sterilize and/or deimmunize the object. In one example, object 12 may be a gastrointestinal scope, e.g., duodenoscope 340, FIG. 17, endoscope 342, FIG. 18 or colonoscope 344, FIG. 19, similar type gastrointestinal scope, or any object discussed above that will be disposed inside positioning and/or storage device 550, and/or a lumen or biofilm on the lumen as discussed above. In one design, positioning and/or storage device 54 includes at least one area of a dielectric material, e.g., area 552 of dielectric material, configured to focus the microwaves provided by electromagnetic device at a predetermined area of object 12, e.g., the area of object 12 indicated at 554 to further increase proteolysis and irreversible destruction to sterilize and/or deimmunize object 12 and enhanced drying of non-toxic aqueous solvent 304 at predetermined area 554 of object 12. In one example, the dielectric material of area 554 may include glass, polytetrafluoroethylene (PTFE), polypropylene, or similar type materials.

Positioning and/or storage device 550 may also include susceptor 560 which is couples to the dielectric material in area 552, e.g., as shown by arrow 562, to further focus the microwaves provided by the electromagnetic device at predetermined area 554 of object 12 to further increase proteolysis and the irreversible destruction of proteins to sterilize and/or deimmunize the object and enhance drying of the non-toxic aqueous solvent on object 12.

In one design, positioning and/or storage device 550 may include electromagnetic shield 570 positioned in a predetermined area of positioning and/or storage device 550, e.g., as shown by arrow 572, configured to house a predetermined portion of object 12, e.g., portion 574 of object 12, such as the control head or any part of a gastrointestinal scope, e.g., a duodenoscope, endoscope, colonoscope, or similar type gastrointestinal scope, or any lumen of any medical, surgical, veterinary device or any object or thing that has a lumen that needs to be sterilized and/or deimmunized and includes electronics which may be sensitive to microwaves or the electromagnetic radiation provided by electromagnetic device. Electromagnetic shield 570 minimizes microwaves from contacting and damaging portion 572 of object 12.

In one design, positioning and/or storage device 550 includes tray 576 and cover 578. Cover 578 may include one or more ports 310, similar to ports 310 discussed above with reference to FIGS. 11 and 12, configured to apply the non-toxic aqueous solvent in or on object 12. In one design, positioning and/or storage device 550 may include one or more openings, e.g., openings 580, which in this example, are included in cover 578, although openings 580 may be located in tray 576, configured to receive one or more of fluid lines coupled to solvent delivery subsystem 302, e.g. one or more fluid lines 324, 326, and/or 328, FIG. 13, and inject the non-toxic aqueous solvent 304 to a lumen of the object, as indicated at 582. As discussed above, the lumen may include the lumen of a gastrointestinal scope, e.g., lumen 346, FIG. 17, of duodenoscope 340, lumen 348, FIG. 18, of endoscope 342, lumen 350, FIG. 19, of colonoscope 344, or similar type lumen of any medical, surgical, dental, veterinary device or any object or thing that has a lumen that needs to be sterilized and/or deimmunized.

In one design, positioning and/or storage device 554 is configured to store a sterilized and/or deimmunized object 12 in sterilize condition for a predetermined amount of time, e.g., days to months.

The proteins of infectious agents and/or immunological agents in or on object irreversibly destroyed by system 300 and the method therein to efficiently and effective sterilized and/or deimmunize object 12 are preferably components of infectious and/or immunological agents including spore forming bacteria, vegetative bacteria, viruses, funguses, mix of bacteria protected by a biofilm, infectious or immunological proteins, including prions.

In one example, electromagnetic device in the chamber is configured as a modified microwave device, e.g., in one example, the heat sinks of a microwave oven may be removed to provide a modified microwave device.

### EXAMPLE 3

### Wet/Dry Cycle

### Example A

Testing was conducted using the prototype of system 300 discussed above with reference to one or more of FIGS. 11-24 to determine the ability of system 300 and the method thereof to sterilize and/or deimmunize object 12 using small cores of sponge material 330, FIG. 16, suspended in small tubes 330, e.g., a 0.5 ml polypropylene tubes, and inoculated with infectious spores. Preferably, sponge material 330 has minimal conductivity when wet or dry. One example of the sponge material 330 may be a felted, hydrophilic, polyurethane foam, e.g., disclosed in U.S. Pat. Nos. 6,855,741 B2 and 6,841,586 B2, incorporated by reference herein.

To fit the sponge fragments into 0.5 ml polypropylene tubes, sponge cores are cut approximately 0.5 cm across and 0.5 cm thick, e.g. as shown in FIG. 16. The sponge cores are suspended in the polypropylene tubes, inoculated with infectious agents and dried. When ready to use, the tubes 330 are modified by cutting the bottom off before sterilization experiments, e.g., indicated at 334. This design of the sample mimics proteins of infectious agents and/or immunological agents inside a lumen of any medical, surgical, dental, veterinary device or any object or thing that has a lumen that needs to be sterilized and/or deimmunized. The inoculated sponges in the polypropylene tubes include 1×10⁶ G. stearothermophilus spores and samples were dried, e.g., overnight. The temperature of chamber 14 was maintained by temperature control subsystem 356, shown in one of more of FIGS. 11-24, to range between about 90° C. to about 100° C. The samples were placed in a polypropylene rack, e.g., polypropylene rack 580, FIGS. 14 and 15, and about 25 ul. of non-toxic aqueous solvent 304 was added by fluid delivery subsystem 302, e.g., peristaltic pump 320, FIG. 13. Non-toxic aqueous solvent 304 included ethyl alcohol and water mixtures of 70%, 60%, 50%, 40%, 30%, 20%, 10%, 0%. Protein conditioning was provided by the wet/dry cycles using controller subsystem 40 discussed in above with reference to one of more of FIGS. 11-24 for about 90 sec each for a total time of 2 hours. Within each wet/dry cycle, the load (the percent of cycle in which electromagnetic device is activated) ranged from about 15% and to 100%. About every 10 to 15 minutes, 25 ul. of solvent was added by solvent delivery subsystem 302 to samples to rewet the samples.

### Example B

Testing was conducted using the prototype of system 300 discussed above with reference to one or more of FIGS. 11-24 to determine the ability of system 300 and the method thereof to sterilize and/or deimmunize object 12 using small cores of sponge material 330, FIG. 16, suspended in small tubes 330, e.g., a 0.5 ml polypropylene tubes, and inoculated with infectious spores. Preferably, sponge material 330 has minimal conductivity when wet or dry. One example of the sponge material 330 may be a felted, hydrophilic, polyurethane foam, e.g., disclosed in U.S. Pat. Nos. 6,855,741B2 and 6,841,586B2, incorporated by reference herein.

To fit the sponge fragments into 0.5 ml polypropylene tubes, sponge cores are cut approximately 0.5 cm across and 0.5 cm thick, e.g. as shown in FIG. 16. The sponge cores are suspended in the polypropylene tubes, inoculated with infectious agents and dried. When ready to use, the tubes 330 are modified by cutting the bottom off before sterilization experiments, e.g., indicated at 334. This design of the sample mimics proteins of infectious agents and/or immunological agents inside a lumen of any medical, surgical, dental, veterinary device or any object or thing that has a lumen that needs to be sterilized and/or deimmunized. The inoculated sponges in the polypropylene tubes including 1×10⁶ G. stearothermophilus spores and samples were dried, e.g., overnight. The temperature of chamber 14 was maintained by temperature control subsystem 356, shown in one of more of FIGS. 11-24 to range between about 60° C. to about 70° C. The samples were placed in a polypropylene rack, e.g., polypropylene rack 580, FIGS. 14 and 15, and about 50 ul. of non-toxic aqueous solvent 304 was added by fluid delivery subsystem 302, e.g., peristaltic pump 320, FIG. 11 Non-toxic aqueous solvent 304 included ethyl alcohol and water mixtures of 70%, 60%, 50%, 40%, 30%, 20%, 10%, 0%. Protein conditioning was provided by the wet/dry cycles using controller subsystem 40 discussed in above with reference to one of more of FIGS. 11-24 for about 90 sec each for a total time of 2 hours. Within each wet/dry cycle, the load (the percent of cycle in which electromagnetic device is activated) ranged from about 15% and to 100%. About every 10 to 15 minutes, 25 ul. of solvent was added by solvent delivery subsystem 302 to samples to rewet the samples.

### Example C

Testing was conducted using the prototype of system 300 discussed above with reference to one or more of FIGS. 11-24 to determine the ability of system 300 and the method thereof to sterilize and/or deimmunize object 12 using small cores of sponge material 330, FIG. 16, suspended in small tubes 330, e.g., a 0.5 ml polypropylene tubes, and inoculated with infectious spores. Preferably, sponge material 330 has minimal conductivity when wet or dry. One example of the sponge material 330 may be a felted, hydrophilic, polyurethane foam, e.g., disclosed in U.S. Pat. Nos. 6,855,741B2 and 6,841,586B2, incorporated by reference herein.

To fit the sponge fragments into 0.5 ml polypropylene tubes, sponge cores are cut approximately 0.5 cm across and 0.5 cm thick, e.g. as shown in FIG. 16. The sponge cores are suspended in the polypropylene tubes, inoculated with infectious agents and dried. When ready to use, the tubes 330 are modified by cutting the bottom off before sterilization experiments, e.g., indicated at 334. This design of the sample mimics proteins of infectious agents and/or immunological agents inside a lumen of any medical, surgical, dental, veterinary device or any object or thing that has a lumen that needs to be sterilized and/or deimmunized. The inoculated sponges in the polypropylene tubes including 1×10⁶ G. stearothermophilus spores and samples were dried, e.g., overnight. The temperature of chamber 14 was maintained by temperature control subsystem 356, shown in one of more of FIGS. 11-24, to range between about 60° C. to about 70° C. or about 90° C. to about 100° C. A prewash cycle was conducted by placing samples in a polypropylene rack, e.g., polypropylene rack 580, FIGS. 14 and 15, and adding about 25-50 ul. of hydrogen peroxide solution ranging from 10-35% and the samples were dried for about 20 minutes in a 100% load. A sterilization reaction was initiated by adding 25 ul. of non-toxic aqueous solvent 304 by fluid delivery subsystem 302, e.g., peristaltic pump 320, FIG. 13. Non-toxic aqueous solvent 304 included ethyl alcohol and water mixtures of 70%, 60%, 50%, 40%, 30%, 20%, 10%, 0%. Protein conditioning was provided by the wet/dry cycles using controller subsystem 40 discussed in above with reference to one of more of FIGS. 11-24 for about 90 sec each for a total time of 2 hours. Within each wet/dry cycle, the load (the percent of cycle in which electromagnetic device is activated) ranged from about 15% and to 100%. About every 10 to 15 minutes, 25 ul. of solvent was added by solvent delivery subsystem 302 to samples to rewet the samples.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

What is claimed is:

1. A system for sterilizing and/or deimmunizing an object, the system comprising:
a stationary chamber at ambient pressure configured to store an object to be sterilized and/or deimmunized therein;
a solvent delivery subsystem coupled to the chamber configured to apply a directed volume of a non-toxic aqueous solvent to coat and wet the object to optimally hydrate proteins of infectious agents and/or immunological agents in or on the object for proteolysis;
an electromagnetic device coupled to the chamber configured to direct microwaves at the object to induce proteolysis of the proteins and generates heat to dry the non-toxic aqueous solvent in or on the object in or on the object;
a temperature control subsystem coupled to the chamber configured to control the temperature of the chamber to induce a temperature of the proteins which accelerates proteolysis of the proteins and dries the non-toxic aqueous solvent in or on the object; and
a controller subsystem coupled to the solvent delivery subsystem, the electromagnetic device and the temperature control subsystem configured to provide a wet/dry cycle including activating the solvent delivery subsystem for a predetermined amount of time to wet and hydrate the proteins for proteolysis, activating the electromagnetic device for a predetermined amount of time to induce proteolysis of the proteins and dry the non-toxic aqueous solvent in or on the object, and activating the temperature control subsystem a predetermined amount of time to accelerate the proteolysis of the proteins and dry the non-toxic aqueous solvent in or on the object, and repeating the wet/dry cycle a predetermined number of times to irreversibly destroy proteins in or on the object to sterilize and/or deimmunize the object.

2. The system of claim 1 in which the temperature control subsystem is configured to maintain a temperature in the chamber which does not damage the object.

3. The system of claim 1 in which the object includes a gastrointestinal scope.

4. The system of claim 3 in which the gastrointestinal scope includes one of: a duodenoscope, an endoscope, or a colonoscope.

5. The system of claim 4 in which the object includes a lumen in the duodenoscope, the endoscope, or the colonoscope.

6. The system of claim 5 in which the object includes a biofilm on the lumen.

7. The system of claim 4 in which the object includes a movable tip of the duodenoscope.

8. The system of claim 1 in which the electromagnetic device is activated a predetermined amount of time to heat the chamber, the object, and the proteins to a predetermined range of temperatures.

9. The system of claim 1 in which the temperature control subsystem includes one or more heating devices coupled to the chamber configured to heat the chamber, the object, and the proteins to a predetermined range of temperatures.

10. The system of claim 1 further including one or more cooling devices coupled to the chamber configured to cool the chamber, the object, and the proteins to a predetermined range of temperatures.

11. The system of claim 1 further including a positioning and/or storage device inside the chamber configured to secure and position the object in the positioning and/or storage device to increase the proteolysis and the irreversible destruction of proteins in or on the object to sterilize and/or deimmunize the object.

12. The system of claim 11 in which the positioning and/or storage device includes at least one area of a dielectric material positioned proximate a predetermined area of the object, the dielectric material configured to focus the microwaves at the predetermined area of the object to further increase the proteolysis and the irreversible destruction of proteins to sterilize and/or deimmunize the object and to enhance drying the non-toxic aqueous solvent at the predetermine area of the object.

13. The system of claim 12 further including a susceptor coupled to the dielectric material configured to further focus the microwaves at the predetermined area of the object to further increase the proteolysis and the irreversible destruction of the proteins to sterilize and/or deimmunize the object and enhance drying of the non-toxic aqueous solvent in or on the object.

14. The system of claim 11 in which the positioning and/or storage device includes an electromagnetic shield housing configured to house a predetermined portion of the object and minimize microwaves from contacting and damaging the portion of the object.

15. The system of claim 11 in which the positioning and/or storage device includes a tray for securing and positioning the object and a cover.

16. The system of claim 15 in which the cover includes one or more ports coupled to the solvent delivery subsystem configured to apply the non-toxic aqueous solvent in or on the object.

17. The system of claim 11 in which the positioning and/or storage device includes one or more openings configured to receive a line coupled to the solvent delivery system, the line configured to apply the non-toxic aqueous solvent to a lumen of the object.

18. The system of claim 17 in which the lumen of the object includes the lumen of a duodenoscope, an endoscope, or a colonoscope.

19. The system of claim 18 in which the object includes a biofilm on the lumen.

20. The system of claim 11 in which the positioning and/or storage device is configured to store a sterilized and/or deimmunized object in a sterile condition for a predetermined amount of time.

21. The system of claim 1 in which the non-toxic aqueous solvent includes one or more of: water, an alcohol solution, hydrogen peroxide, an ionic detergent, and/or a non-ionic detergent.

22. The system of claim 1 in which the proteins are components of an infectious and/or immunological agents including spore forming bacteria, vegetative bacteria, viruses, funguses, a mix of bacteria protected by a biofilm, infectious or immunological proteins, and toxic proteins.

23. The system of claim 1 in which the electromagnetic device and the chamber is configured as a modified microwave device.

24. A method for sterilizing and/or deimmunizing an object, the method comprising:
providing a stationary chamber at ambient pressure configured to store an object to be sterilized and/or deimmunized therein;
applying a directed volume of a non-toxic aqueous solvent to coat and wet the object to optimally hydrate proteins of infectious agents and/or immunological agents in or on the object for proteolysis;
directing microwaves at the object to induce proteolysis of the proteins and generate heat to dry the non-toxic aqueous solvent in or on the object;
controlling the temperature of the chamber such that the temperature of the proteins in or on the object accelerates proteolysis of the proteins in or on the object and dries the non-toxic aqueous solvent in or on the object; and
providing a wet/dry cycle including coating and wetting the proteins to optimally hydrate the proteins applying microwaves a predetermined amount of time to induce proteolysis of the proteins and dry the non-toxic aqueous solvent in or on the object, controlling the temperature a predetermined amount of time to accelerate proteolysis of the proteins and dry the non-toxic aqueous solvent in or on the object, and repeating the wet/dry cycle a predetermined number of times to irreversibly destroy proteins in or on the object to sterilize and/or deimmunize the object.

25. The method of claim 24 in which controlling the temperature includes maintaining a temperature in the chamber which does not damage the object.

26. The method of claim 24 in which the object includes a gastrointestinal scope.

27. The method of claim 25 in which the gastrointestinal scope includes one of:
a duodenoscope, an endoscope, or a colonoscope.

28. The method of claim 25 in which the object includes a lumen in the duodenoscope, the endoscope, or the colonoscope.

29. The method of claim 27 in which the object includes a movable tip of the duodenoscope.

30. The method of claim 28 in which the object includes a biofilm on the lumen.

31. The method of claim 24 in which controlling the temperature of the chamber includes providing a predetermined range of temperatures in the chamber.

32. The method of claim 24 in which directing the microwaves includes focusing the microwaves on a predetermined area of the object.

33. The method of claim 24 including minimizing the microwaves from contacting a portion of the object.

34. The method of claim 24 in which the non-toxic aqueous solvent includes one or more of: water, an alcohol solution, hydrogen peroxide, an ionic detergent, and/or a non-ionic detergent.

35. The method of claim 24 in which the proteins are components of an infectious and/or immunological agents including spore forming bacteria, vegetative bacteria, viruses, funguses, a mix of bacteria protected by a biofilm, infectious or immunological proteins, and toxic proteins.
